(19) United States
(12) Patent Application Publication
(10) Pub. No.: US 2016/0199576 A1
(43) Pub. Date: Jul. 14, 2016

(54) WEARABLE INTRAVENOUS FLUID DELIVERY SYSTEM

(71) Applicant: Jacqueline Sarah SAVAGE, Abbotsford, Victoria (AU)

(72) Inventor: Jacqueline Sarah Savage, Abbotsford Victoria (AU)

(21) Appl. No.: 14/915,818

(22) PCT Filed: Sep. 3, 2014
(86) PCT No.: PCT/AU2014/000872

§ 371 Date: Mar. 1, 2016

(30) Foreign Application Priority Data
Sep. 3, 2013 (AU) 2013903357

(51) Int. Cl. A61M 5/172; A61M 5/14

(52) U.S. Cl. CPC A61M 5/1723; A61M 5/1415; A61M 2005/1416; A61M 2230/005; A61M 2230/50; A61M 2230/04; A61M 2230/40; A61M 2230/20; A61M 2205/50

(57) ABSTRACT

A garment and system for providing intravenous fluid delivery to a patient is provided. The garment is worn adjacent to the skin of the patient and includes a pump unit support portion to support a portable infusion pump and a fluid reservoir support portion to support a fluid reservoir carrying fluid for intravenous delivery to the patient via the infusion pump.

The present application claims priority from Australian Provisional Patent Application No 2013903357 titled “WEARABLE INTRAVENOUS FLUID DELIVERY SYSTEM” and filed on 3 Sep. 2013, the content of which is incorporated by reference in its entirety.

### TECHNICAL FIELD

Described embodiments relate generally to wearable aids for intravenous fluid delivery and systems and methods employing such aids. Some embodiments employ one or more sensors to sense at least one biological condition of the patient wearing the wearable aid.

### BACKGROUND

Cancer is the leading cause of death worldwide, contributing to approximately 7.6 million deaths per annum (around 13% of all deaths). It is reported that in Australia, the likelihood of being diagnosed with cancer prior to the age of 85 is one in two for males and one in three for females. In 2011, over 43,700 deaths were directly attributed to cancer and its related illnesses, with associated health system costs exceeding more than $3.8 AUD billion. The number of newly diagnosed cancer cases every year in Australia alone is over 130,000, of which over 50% of patients receive chemotherapy as their primary treatment method.

Conventional chemotherapy treatment often presents complications, such as adverse physical side effects, and is an intrusive treatment delivery method, resulting not only in physical discomfort for the patient but also psychological distress. These issues considerably reduce a patient's ability to carry out daily activities commonly resulting in reduced independence and severity of their health condition, impinging on recovery rates. Furthermore, the primary side effect experienced by patients receiving chemotherapy is that their immune response is suppressed, which makes them susceptible to infection. In a study by Creutziug, an industry expert, it was found that infection is the primary cause of death for patients receiving chemotherapy treatment (eliminating disease progression fatalities).

Research has revealed that the use of a continuous low dose infusion of the chemotherapy drugs has proven to alleviate the side effects experienced during the treatment (Orlando L, et al. 2006). The slow infusion method increases the time in which the drug is administered from the current 4-6 hour intensive treatment to a 96 hour infusion cycle. In addition, it has been observed that the slow infusion method has generated a greater cellular response to the chemotherapy drugs, resulting in a more effective treatment method (Kerbel, R. S. et al. 2002).

Portable infusion pump devices may be used to deliver the chemotherapy drugs. Common complaints about current portable infusion pump devices vary from noise of the machine to the inability to comfortably wear the product during the day when carrying out daily tasks. Due to the noise and uncomfortable or clumsy product configuration of many current pump devices, sleeping with the device is undesirable. Patients have reported that the portable pump has improved their attitudes towards the treatment as it has provided them with greater autonomy; however the current pump designs limit the portability and comfort of the comfort of such pumps due to their large size. In addition, these existing devices weigh an average of 700 grams including drug fluid, further hindering patient comfort. Current pumps have also been reported to be difficult to programme as well as providing no feedback to the patient of the treatment's progress, which may affect pump efficacy and patient reassurance (Bergeson, B. 2010). Furthermore, the inability to wear these products comfortably whilst sleeping, results in a limitation on the time the chemotherapy drugs can be delivered into the body, consequently not maximizing the full potential of the continuous low dose release system (Orlando, L. et al. 2006).

It is desired to address or ameliorate one or more shortcomings or disadvantages of prior intravenous fluid delivery techniques, or to at least provide a useful alternative thereto.

### SUMMARY

In a first aspect the present invention accordingly provides a garment for providing intravenous fluid delivery to a patient, the garment operable to be worn adjacent to the skin of the patient and including:

a pump unit support portion to support a portable infusion pump; and

a fluid reservoir support portion to support a fluid reservoir carrying fluid for intravenous delivery to the patient via the infusion pump.

In another form, the garment is sized and shaped to be tight-fitting to be wearable under other garments.

In another form, the garment is configured to be worn on an upper body of the patient.

In another form, the garment is configured as a vest.

In another form, the pump unit support portion is located to be on a front of the upper body.

In another form, the fluid reservoir support portion is located on a front or back of the upper body.

In another form, if the fluid reservoir support portion is located on a back of the upper body, the fluid reservoir support portion is positioned to overlie one of the thoracic spine and the lumbar spine, and if the fluid reservoir support portion is located on a front of the upper body, the fluid reservoir support portion is positioned to overlie one or more of the abdomen and the thorax.

In another form, the garment includes at least one biological sensor carrying portion for carrying a biological sensor operative to measure a biological condition of the patient.

In another form, the at least one biological sensor carrying portion is for carrying a sensor to sense at least one of: temperature; heart rate; pulse; respiratory rate; electrocardiogram signals; respiratory noise; blood pressure and blood oxygen saturation.

In another form, the at least one biological sensor carrying portion includes a plurality of biological sensor carrying portions, the plurality of carrying portions arranged to carry respective temperatures sensors to in combination sense a core temperature of the patient.

In another form, the locations on the garment of the plurality of biological sensor carrying portions are selected from:
either side of the rib cage under the arm;
sternum; or
upper thoracic region.

In another form, the garment is at least partly stretchable and comprises moisture transmissive materials in at least some parts of the garment that are to overlie the skin.

In a second aspect the present invention accordingly provides a system for providing intravenous fluid delivery to a patient, comprising:

a garment worn by the patient adjacent to the skin, the garment including:
a pump unit support portion supporting a portable infusion pump; and
a fluid reservoir support portion supporting a fluid reservoir, wherein the fluid reservoir is operably connected to the portable infusion pump by a fluid supply conduit to pump fluid from the fluid reservoir for intravenous delivery to a delivery site.

In another form, the garment is sized and shaped to be tight-fitting to be wearable under other garments.

In another form, the garment is configured to worn on an upper body of the patient.

In another form, the garment is configured as a vest.

In another form, the fluid supply conduit is incorporated into the garment.

In another form, the system further comprises at least one biological sensor for sensing a biological condition of the patient, the at least one biological sensor carried by a respective biological sensor carrying portion forming part of the garment.

In another form, the at least one sensor is for sensing at least one of: temperature; heart rate; pulse; respiratory rate; electrocardiogram signals; respiratory noise; blood pressure and blood oxygen saturation.

In another form, the garment includes a plurality of biological sensor carrying portions, the plurality of biological sensor carrying portions arranged to carry respective temperatures sensors to in combination sense a core temperature of the patient.

In another form, the locations of the plurality of biological sensor carrying portions are selected from:

either side of the rib cage under the arm;

sternum or

upper thoracic region.

In another form, sensing and status information from the at least one biological sensor is wirelessly or directly communicated to any one of:

a controller for monitoring and controlling the operation of the portable infusion pump;

a transceiver device; or

a handheld computing device.

In another form, any one of the controllers for the portable infusion pump, the transceiver device or the handheld computing device, having received the sensing and status information from the at least one biological sensor, is configured to then determine an alarm condition based on this sensing and status information.

In another form, the alarm condition indicates any one of variation in body temperature, heart rate or respiratory noise level of the patient.

In another form, the alarm condition is further transmitted to another device or system.

In another form, the controller for the portable infusion pump is incorporated into a housing of the portable infusion pump.

In another form, the portable infusion pump is ergonomically designed to conform to a body shape of the patient.

In a third aspect the present invention accordingly provides a method of intravenous fluid delivery, comprising:

fitting a garment to lie adjacent to the skin of a patient to receive the intravenous fluid delivery, the garment carrying a portable infusion pump and a fluid reservoir comprising a volume of fluid to be delivered to the patient; and

controlling the fluid delivery pump to deliver fluid from the fluid reservoir to the patient via an intravenous delivery line.

In another form, the method further comprises:

monitoring at least one biological condition of the patient using at least one sensor coupled to or carried by the garment; and

wirelessly notifying medical personnel of the monitored at least one biological condition.

In another aspect there is provided an infusion pump unit for the measured delivery of fluid to a fluid delivery site, the pump unit comprising:

a pump mechanism arranged to convey fluid through a conduit toward the fluid delivery site;

a wireless communication subsystem to communicate with a computing device over a network; and

sensor output receiving circuitry to receive sensor output signals from at least one biological condition sensor in communication with the pump unit;

wherein the pump unit is configured to transmit data indicative of the sensor output signals to the computing device.

In another form, the wireless communication subsystem comprises a node in a personal area network (PAN) or a body area network (BAN) and is in communication with at least one device in the PAN or BAN.

In another form, the at least one sensor forms part of the PAN or BAN.

In another form, the computing device comprises a node of the PAN or BAN and the computing device is configured to transmit data indicative of the sensor output signals to a remote computing device over a local area network or a public wireless network.

In a further aspect there is provided a pumping assembly for pumping fluid through a continuous fluid supply conduit, the pumping assembly including:

a pump drive arrangement; and

a peristaltic pump mechanism driven by the pump drive arrangement to pump fluid through the continuous fluid supply conduit, wherein the peristaltic pump mechanism is removably coupled from the pump drive arrangement to attach the fluid supply conduit to the peristaltic pump mechanism to.

In another form, the pump drive arrangement includes a housing having a pump mechanism receiving region to receive the peristaltic pump mechanism.

In another form, the peristaltic pump mechanism includes a rotating member to peristaltically pump fluid through the continuous fluid supply conduit.

In another form, the pump drive arrangement rotationally drives a shaft, the shaft adapted to couple with the rotating member of the peristaltic pump mechanism.

In another form, wherein the peristaltic pump mechanism includes:

a first component to which the fluid supply conduit is attached; and

a complementary second component incorporating the rotating member which when combined with the first component will on rotation of the rotating member peristaltically move fluid through the fluid supply conduit.

In another form, on attachment of the fluid supply conduit to the first component, the fluid supply conduit traces an arcuate path.

In another form, the rotating member includes at least one roller that on rotation of the rotating member rolls along the fluid supply conduit to peristaltically move fluid along the fluid supply conduit.

In another form, the fluid supply conduit is clipped into the second component.

In another form, the housing includes a channel portion to receive the fluid supply conduit.

### BRIEF DESCRIPTION OF DRAWINGS

Illustrative embodiments will be discussed with reference to the accompanying drawings wherein:

FIG. 1 is a perspective view of an example system comprising a wearable aid for intravenous fluid delivery;

FIG. 2A is a front view of the wearable aid of FIG. 1, shown when worn by a patient and when coupled to an intravenous fluid delivery line;

FIG. 2B is a side view of the wearable aid of FIG. 1, shown when worn by a patient and when coupled to an intravenous fluid delivery line;

FIG. 3 is an exploded perspective view of a pump unit for use in the wearable aid of FIG. 1:

FIG. 4 is a block diagram schematically illustrating components of the pump unit of FIG. 3;

FIGS. 5A, 5B and 5C are front, back and side views, respectively, of the wearable aid of FIG. 1, illustrating example positions of sensors:

FIG. 6 is a schematic cross-sectional view of an example temperature sensor for use with the wearable aid:

FIG. 7 is a block diagram schematically illustrating coupling of the pump unit to a programming interface to allow fluid delivery settings to be programmed into the pump unit;

FIG. 8 is a perspective view of a garment of the wearable aid, illustrating example fastening locations of the garment;

FIG. 9 is a flowchart of a method of conducting intravenous fluid delivery using the wearable aid;

FIG. 10A is a perspective view of another example system comprising a wearable aid for intravenous fluid delivery,

FIG. 10B is a front view of the wearable aid of FIG. 10A, shown when worn by a patient and when coupled to an intravenous fluid delivery line;

FIG. 10C is a side view of the wearable aid of FIG. 10A, shown when worn by a patient and when coupled to an intravenous fluid delivery line;

FIG. 11 is an exploded perspective view of an example fluid delivery device including a removably attachable pump cassette;

FIG. 12A is a plan view of the pump cassette when partially inserted into a cassette frame for attachment to the fluid delivery device of FIG. 11;

FIG. 12B is a plan view of the pump cassette when fully inserted into the cassette frame of FIG. 12A;

FIG. 12C is an exploded view of the pump cassette illustrated in FIGS. 12A and 12B;

FIG. 13 is a block diagram illustrating a personal area network or a body area network including the pump unit of some embodiments;

FIG. 14 is a front view of a wearable aid for intravenous fluid delivery in accordance with another illustrative embodiment depicting the location of the pump unit;

FIG. 15 is a front perspective view of the wearable aid illustrated in FIG. 14;

FIG. 16 is a front perspective view of the wearable aid illustrated in FIG. 14 with the pump unit inserted and illustrating the adjustable straps;

FIG. 17 is a front perspective view of the wearable aid illustrated in FIG. 16 with the adjustable straps adjusted to the as worn position;

FIGS. 18A to 18D are various views of the pump unit illustrated in FIG. 11 configured to be supported by a wearable aid;

FIG. 19 is an exploded view of the pump unit illustrated in FIGS. 18A to 18E; and

FIG. 20 shows the pump unit illustrated in FIGS. 18A to 18D immediately prior to attachment of the cover,

### DESCRIPTION OF EMBODIMENTS

Described embodiments relate generally to wearable aids for intravenous fluid delivery and systems and methods employing such aids. The fluid delivery may comprise chemotherapy drug delivery or antibiotic delivery, for example. Some embodiments employ one or more sensors to sense at least one biological condition of the patient wearing the wearable aid.

Described embodiments include a portable drug infusion system, including a garment that patients can wear comfortably while receiving treatments over a prolonged period of multiple hours or days. This system has been designed to assist in addressing physical side effects which result from treatments by providing a system that allows a slow, low dose delivery method. In addition, continual monitoring of a patient's vital signs with wireless data feedback to healthcare professionals will allow for early signs of infection to be detected, reducing the risk of death due to infection. The intent of this system is to provide the user with holistic patient care that improves the patients' quality of life by providing greater independence and mobility, improved recovery rates and overall treatment effectiveness.

This disclosure includes a description of various considerations appropriate to intravenous (IV) fluid delivery and in particular to the IV delivery of chemotherapy drugs, antibiotics and other treatment fluids for the treatment of cancer or other conditions. This disclosure focuses firstly on such considerations as context for the detailed description of embodiments and of the drawings, which follows.

Intravenous infusion of chemotherapy drugs is the most commonly used method of delivery. Its preference is due to its ability to provide rapid and reliable delivery of the drugs. However, intravenous delivery of such toxic drugs may irritate the veins, potentially causing venous spasm and pain. Despite this, the method has shown to be the most effective and reliable method of administration. Intravenous therapies may be given through a catheter placed in a vein in the arm or hand (peripheral line) using a cannula. In addition, intravenous drugs may also be given through a catheter placed into a larger vein in the chest or neck, these are known as a central venous catheter (CVC) or central line.

Chemotherapy treatments are most commonly given in regular intervals called cycles. Each cycle may involve a dose of one or more drugs followed by several days or weeks without treatment. In doing this, normal cells are given time to recover from the drug's side effects. In some cases, doses may be administered several days in a row, or every other day for multiple days, after which a period of rest days is allocated. Recent studies of the continuous slow release of the drug into the body over numerous days have shown to alleviate at least some side effects of the drugs as well as improve the body's cellular response to the treatment. When cancer patients are exposed to large doses of toxic chemotherapy drugs in a short period of time, patients must go through periodic lapses in treatment to allow their body time to regain strength. During this period of resting, the body is provided with time to regenerate the blood vessels which tumours require to live, resulting in a cycle of treatment which can be ineffective (Kerbel, R. S. et al. 2002.

Recent research into a treatment method that involves continuous chemotherapy doses administered in small quantities has had very promising results. The new treatment has shifted the focus of chemotherapy from the tumour to the blood vessels that feed the tumour. The low-dose chemotherapy aims to limit the growth of certain blood vessels which supply the tumour with needed nutrients in control, resulting in little or no tumour growth. When low dose chemotherapy is administered on a daily schedule, the continual death of endothelial cells occurs, preventing or limiting new blood vessel formation and substantially disrupting the angiogenic process, slowing down tumour growth rapidly.

Test trials of this new treatment were carried out by oncologists in Milan, who have published long-term responses of patients with breast cancer receiving this slow low dose release treatment (Orlando, L. et al. 2006). The results of the test were positive, with 32% patients achieving either a complete or partial remission. In a further 16% of patients, no tumour growth or progression was recorded in over a year. In cases where tumour progressions did occur, it was observed that the therapy was slowing the spread of the disease. In addition, as the therapy is targeted to attack the cancer's support structure, rather than the cancer itself, it has potential to be applicable for all types of cancer that requires angiogenesis for growth. Further research and patient surveying was conducted, revealing that due to the treatment's slow and low dose administration of the drugs, patients were experiencing minimal side effects, in comparison to conventional chemotherapy high dose/short time treatments. Side effects that were documented consisted of a small minority of patients experiencing a mild suppression of white blood cell count (Orlando, L. et al. 2006). As research continues, the development of new drugs will allow for this method of treatment to be an accessible and viable option for all. Reduced systemic toxicity means that such treatments can also be used in sicker patients, and that they can carry new chemotherapeutic agents that would have been far too toxic to deliver via traditional systemic approaches.

Described systems and methods may use biological monitoring equipment to monitor patients' vital signs, such as core body temperature, blood oxygen levels, pulse and blood pressure, for example. Other biological condition indicators that can be monitored using suitable sensors fitted within or coupled to the garment may allow respiratory rate, ECG signals, respiratory noise or other indicators of short term (acute) conditions. Such monitoring can advantageously increase the rate of infection detection and effectively track patients' treatments and their relative health.

Described embodiments are intended to be more human-centric and user friendly compared to previous intravenous fluid delivery techniques, allowing patients to comfortably execute daily activities as well as sleep while wearing the system and receiving treatment. A system that patients can wear continuously over longer periods of time allows for a slow infusion method of drug administration to be employed, which further reduces side effects and increases the effectiveness of the treatment.

The quality of life of a patient is defined as individual perception of life, values, objectives, standards, and interests in the framework of culture, according to the World Health Organisation (WHO). Quality of life is continuously used in studies as a primary measure in order to evaluate the effectiveness of treatments. In the case of cancer and chemotherapy treatment, the hope of cure is weighed against the certainty of death, resulting in both doctor and patient willingly accepting the toxicity associated with chemotherapy and its impact on the quality of life for the patient (Dehkordi A et al. 2009). The physical health and psychological side effects generated by chemotherapy can be severe, drastically reducing the patient's quality of life. In cases where the cancer is terminal and chemotherapy is prescribed only to prolong the patient's life for a limited amount of time, many patients make the decision to not partake in the treatment as their quality of life is drastically reduced and they would prefer to enjoy their remaining months rather than enduring the side effects that chemotherapy causes.

Recent studies were carried out in Switzerland, where researchers evaluated the impact that chemotherapy being administered in the patients' home, via a portable infusion pump, had on the patients' quality of life. The study showed that patients receiving treatment via a portable system at home experienced a greater quality of life compared to patients receiving treatments in hospital (as in-patients). This result was reflected in the patients' improved sociability, social role and reduced emotional distress. Individuals who received treatment via a portable pump at home reported to be highly satisfied with treatment and none of the patients requested to go into hospital for any of their treatments.

Additional concerns were associated with the potential technical problems of the pump in the case of failure and the side effects of the chemotherapy treatment. Caregivers and relatives were requested to take part in the questionnaires, showing 100% of the patients' support people being in favour of home treatment. Relatives stated they there was better tolerance due to fewer side effects, less distress (90%) and less asthenia—loss of strength (48%). Overall, the results concluded that the use of a portable, programmable pump provided complete autonomy to patients and enhanced satisfaction of the treatment. The major determinants of this conclusion were the higher comfort levels and reassurance of having a relative present (Lüthi F, et al. 2011).

Chemotherapy treatment results in the death of rapidly dividing cells, including tumour cells and healthy cells. Some of the most rapidly dividing cells are bone marrow cells which are responsible for the production of white blood cells, red blood cells and platelets. Thus, chemotherapies are generally considered to be immunosuppressive. In the treatment of cancer using chemotherapy drugs, there is a fine balance between tumour toxicity and general toxicity of the body, and as a result, the patient's blood must be closely monitored and in order to avoid creating a high level of general body toxicity (Weir, G er al., 2011). Consequently, regular blood tests are conducted for patients before every treatment to ensure that patients are being treated within safe parameters.

A Full Blood Count (FBC) is usually conducted the day before chemotherapy treatment; this is done to obtain current and accurate results. A FBC will be conducted to assess the patient's red blood cell count, white blood cell count and platelet count—this is to ensure that levels have not been lowered too much by the last treatment, otherwise future treatments must be held off until a healthy level is resumed. When white blood cell count is lowered there is an increased risk of infection for the patient. Neutrophils are a type of white blood cell which helps the body fight infection. Research has found that infection is the primary cause of death, other than disease progression fatalities, for patients receiving chemotherapy treatment (Creutzig U et al. 2003). Patients with a lowered level of neutrophils (neutropenia) are at high risk of developing a serious infection. Neutropenia is reported to occur in more than 50% of people with cancer who are receiving chemotherapy treatment (Managing side effects, 2012).

When a patient's white blood cell count is decreased due to chemotherapy, it lowers the individual's immune system and the reduced presence of neutrophils means that signs of inflammation can be extremely subtle. This presents a further risk for the patients, since the initial signs of an infection can consequently go unnoticed, making infection detection harder. As a result patients are encouraged to take their body temperature regularly in order to identify the early onset of a fever. Body temperature monitoring is consequently an important aspect to monitoring the patient's health.

Low blood pressure (Hypotension)—below 90/60—is a common side effect of some chemotherapy drugs. In addition, low blood pressure may be a result of low blood count (anemia) caused from chemotherapy treatment or cancer (Blood Pressure Changes, 2013). Blood pressure readings are taken before the commencement of every chemotherapy treatment session, using an electrical monitor and inflatable cuff.

The generally accepted method of detecting oxygen saturation in blood is known as Pulse oximetry, which relates the light absorption characteristics of saturated hacmoglobin to give an indication of oxygen saturation in blood. The resulting Pulse Wave Amplitude (PWA) deviations indicate increases and decreases in arterial oxygen saturation (SaO2), where healthy individuals characteristically have a SaO2 between 97% and 99%. Commonly, arterial oxygen saturation is measured at either the fingertip or the ear lobe using a pulse oximeter. This is particularly important to monitor in patients with respiratory tract cancer such as lung cancer where their respiration rate is compromised by the cancer.

The pulse rate is defined as the rate at which your heart beats. Pulse oximetry is also used to measure this vital sign. Measuring a patient's heart rate or pulse provides very important information about an individual's health. It is one of the most effective ways of identifying potentially abnormal heart rhythms, particularly in patients with cardiac tumours. In addition to killing cancer cells, chemotherapy can also kill other cell types in various organs. Most organs are able to regenerate cells after having been damaged; however heart muscle cells cannot be regenerated. The loss of these cells can weaken the heart muscle, leading to dilated cardiomyopathy, a condition where the heart's pumping action is reduced, potentially resulting in heart failure (Rattue. P, 2012).

An intravenous infusion system is the process by which an infusion device is used to deliver fluids or drugs in solution to the patient by the intravenous route. For optimal infusion, devices must reliably deliver drugs to the patient at pressures which overcome all baseline and intermittent resistance, but cause no harm to the patient. Resistance in an IV circuit exists in the internal diameter of connecting tubing, cannula, needles and the patient's vessel's, which results in a higher pressure required from the IV circuit in order to overcome the resistance and obtain prescribed flow. Consequently, these systems must be capable of delivering infusion pressures of 100-750 mmHg.

Infusion pumps are powered pumps which use a positive pumping action to administer fluid and drugs intravenously into a patient. These systems are able to provide an accurate flow rate of drugs over a specified period of time. There are two modes of pumping action used in these devices, volumetric and syringe drivers. Volumetric pumps use a linear peristaltic or piston pump to control the infusion flow rate and can administer up to 1000 ml of fluid at flow rates of 0.1 to 1000 ml/hr. Syringe drivers utilise an electrically controlled, electric motor to drive a plastic syringe plunger. These pumps administer up to 100 ml of fluids at flow rates of 0.1-100 ml/hr and can be a suitable pump for lower volume and low flow rate infusions.

Ambulatory (portable) infusion pumps can be smaller and lighter and may comprise battery powered syringe or cassette devices. Many of these types of pumps existing on the market have only minimal alarms, thus patients and carers must be vigilant in administration observations. Careful consideration of the portable device needs to be given when exposed to hazards such as knocks, fluids, electro-magnetic interference, etc. Currently, according to some views, critical drugs which require constant flow should not be administered using ambulatory pumps (Davis. W, 2010). Despite advancements in their size, the weight of many existing portable pumps is still reported to be an issue for patients as well as the noise generated by the electric pump. The pump generates a continuous humming sound at an average of 25 dB, causing irritation to the user especially when trying to sleep (Mitchell T 2007).

Non-electronic ambulatory infusion pumps have recently been created by Baxter™. The Baxter Elastomeric Pumps deliver medication to the patient through the use of an elastomeric “balloon” which consistently deflates and pushes solution through the IV tubing and into the patient. These pumps are designed to improve quality of life of the patient by allowing continuous infusion treatment without the inconvenience of programming, power sources and alarms. Drawbacks associated with the non-electronic pump are the inability to carefully control the rate of infusion and pressure. In addition, the absence of safety alarms is not ideal for the infusion of toxic drugs such as chemotherapy drugs. Due to the use of the elastomeric “balloon”, the pump's flow rate is most accurate at 21 C and is recommended to be kept at room temperature, ensuring the device is not exposed to extreme temperatures (Baxter Corporation, 2010). Despite the elimination of the electrical internals, the pump is still not able to be submerged in water or exposed to a direct stream of water. Furthermore, the pump must be carried at a particular height on the patient, making sure the top of the infuser is kept as close to the height where the IV line enters the patient's body.

In addition to chemotherapy treatment delivery, described embodiments can be used for intravenous antibiotics administration. Intravenous antibiotics are medications which are delivered directly into the bloodstream. Commonly, the medication is delivered slowly through a drip process, which helps to avoid introducing air into the blood. IV antibiotics are largely used for the treatment of bacterial infections. Through delivering the antibiotics into the bloodstream directly, they are carried to the site of infection with more speed and efficiently in order to promote an increased rate of healing.

IV antibiotics are usually utilised for severe infections which require fast treatment. Minor bacterial growths are treated using oral antibiotics, which possess fewer side effects and chances for complications. Furthermore, IV antibiotics have the added advantage of being able to be administered in much higher doses, depending on the severity and nature of infection being treated. In some cases, intravenous antibiotics may be used in the case of a less severe infection if oral medications cannot get to the appropriate location. Being able to provide patients with a portable drug delivery system with vitals monitoring will not only improve quality of life for the patient but can also reduce medical costs (Balaquer A, et al. 2012). Due to the elimination of overnight stays required in hospital over a period of days up to a few weeks, medical costs are significantly reduced. However, with described embodiments that employ sensors to monitor biological conditions of the patient, vital signs of the patient can be continuously monitored by professionals and the quality of health care can remain uncompromised.

The recent discovery of a new slow, low dose delivery method of chemotherapy has shown to not only alleviate physical side effects of the treatment but also increase the body's cellular response to the drugs, making it a more effective treatment method for cancer and additional chemotherapy treated diseases (Kerbel R S. et al. 2002). In order to allow for the administration of this newly discovered treatment method, the IV fluid delivery system of described embodiments need to provide infusion of drugs at a slow, controlled rate over a sustained period of time, such as at least 24 hours, possibly around 36 hours, 48 hours or possibly a duration of 96 hours or more.

The AS/NZS 3770:1993 Standard is mainly focused on the safe use of stationary infusion pumps in hospitals, although there are included requirements for portable (ambulatory) pumps used in the home. Many of the requirements for stationary infusions remain pertinent for portable applications. In summary:
Life-saving drugs which require infusion are monitored by a pump or controller to ensure accuracy of delivery.
The availability of a “keep vein open” function which is activated at the end of infusion of a pre-set volume of fluid is useful in preventing occlusion due to the coagulation of blood.
Delivery rate can be selected by the operator, usually in increments of 1 mL/h or 1 drop/min.
Rate consistency is required to ensure fluid to be delivered is maintained at a constant rate so as to maintain a specific drug concentration in patient's blood.
Volumetric accuracy is required so that a specific volume of fluid is delivered in a specific unit of time.
Alarms are required for the following conditions: occlusion, air-in-line, low battery, mechanical or electrical faults, low or excessive rate of infusion and infusion complete.
Equipment displays should show set rates continuously. The following operational parameters and conditions may be displayed intermittently or continuously: volume limit, volume infusion, the alarm indicators, the operational status, the current power source, whether the battery is being changed, and intravascular pressure at the infusion site.

The AS/NZS 3200.2.24:1999 Standard specifics the requirement for infusion pumps, infusion controllers, syringe pumps and pumps for ambulatory use. These devices are intended for use by medical staff and home patients as prescribed and medically indicated.

In summary:
Mechanical strength, equipment shall not present a safety hazard to the patient as a result of external vibration.
Remote parts including mains operated adapters and parts shall not present a safety hazard as a result of a free fall from a height of 1 m onto a hard surface. Subsequent to the fall of the remote part, when the equipment is turned on, it shall either function normally or cease delivery and activate alarm.
The equipment shall be designed so that, taking into consideration ageing and rough handling of the equipment, in the event of spillage no liquid is retained within the equipment enclosure and the equipment shall either continue to function normally or cease delivery and activate an alarm.
Equipment shall be so constructed that liquid which might leak from containers, tubing, coupling and the like does not impair safe functioning of the equipment nor wet uninsulated live parts or electrical insulation.
Means shall be provided to prevent over infusion. An audible alarm shall be initiated and shall either cease delivery of infusion or reduce delivery rate.
Audible alarm shall be able to produce a sound-pressure level of at least 65 dB at 1 m.
Ambulatory pumps shall include an alarm if the equipment is switched to a standby mode of operation for more than 1 hr.

Example pump units of described embodiments may meet the above requirements. While some embodiments are aimed at treating IV chemotherapy patients and at addressing their requirements and preferences, it is envisioned that certain aspects of the design could be used in other intravenous drug delivery applications. Such applications would include intravenous delivery of antibiotics, which involves continuous drug infusion over multiple days and necessary vitals monitoring. Embodiments may also be employed for portable continual vitals monitoring for patients at risk of infection or cardiovascular dysfunctions. In such embodiments, the intravenous fluid delivery device may be detached and the wearable aid (with integrated sensors and a wireless transceiver device) may then act primarily as a portable vitals monitoring system in communication with a handheld computing device within a personal area network of the patient wearing the wearable aid.

It is a common view amongst medical professionals that continual monitoring of chemotherapy patients' vitals, in particularly core body temperature, is important in providing optimal care for the patient. Currently, patients are required to monitor their own temperature daily (as a minimum) and due to the side effects of the chemotherapy drug on the users' cognitive ability, as many as 70% of patients forget to measure their temperature as often as required. Furthermore, it should be noted that the rate of infection contracted in patients whilst receiving treatment (due to lowered immunity from chemotherapy drugs) has been recorded as 80%. Patients who reported to not experience any infections or additional illnesses whilst receiving treatment stated that they did not leave their house very often and stayed home for the majority of the time they were receiving treatment. Infections that were readily contracted included, colds, influenza (flu), pneumonia, bronchitis and shingles. In addition, interviewed patients stated that having their vitals continuously monitored and relayed back to their healthcare professionals would not only give them greater trust in the success of their treatment and healthcare, but would also alleviate some of the stress associated with the treatment.

Interviews conducted with oncology nurses revealed unforeseen design complications with existing portable chemotherapy infusion devices. Whilst the average time to set up these products was 15 minutes, the time taken to programme and add additional safety features accounted for majority of the set up time. This may be mitigated with the design of a simpler product interface with intuitive controls and product display.

Current portable IV delivery systems have been reported to create a sense of embarrassment for patients due to the bulkiness, unappealing aesthetics and obviousness of the product when in public. Reducing the stigma associated with chemotherapy treatment can be achieved through a more discreet appearance of the chemotherapy drug delivery system.

Described embodiments include the use of a new portable intravenous (IV) infusion system for the delivery of Chemotherapy treatment (and possibly other treatments) with features incorporated to aid health monitoring and allow for slow low dose infusion delivery to the patient. However, described embodiments may be used for infusions that do not involve slow low dose treatments and instead provide treatment over one to several hours.

The design of the IV fluid delivery system and wearable aid take into account practical considerations, such as cleaning, reducing patient interference or maintenance, and material selection, including hypoallergenic characteristics. Materials usable to construct the wearable aid to substantially resemble a piece of clothing include, by way of example, e-textiles (electronic textiles), Gore-tex™ fabrics, NEXAR™ polymer fabrics, Spandex (elastane), nylon fabrics, polyester fabrics and cotton fabrics. Other fabrics usable as clothing fabrics can be employed as well, including suitable light-weight, breathable (ie moisture transmissive) and elastic fabrics.

Patients, many of which will be over the age of 45, may have degraded dexterity in their fingers and hands and user controls are designed with human factors to that effect in mind. Side effects due to tumour presence, such as pain and discomfort around cancer growths are existent in some users and wearable systems and harnessing devices should be designed according to these conditions. Furthermore, users whom have received surgery and radiation as addition treatments regularly possess painful or uncomfortable wounds on their body. The garment of described embodiments may therefore be configurable to adopt one of a number of possible different configurations in order to allow a configuration that is least irritating to be adopted for a specific patient.

As a medical product, the infusion system described herein is intended to conform to all relevant standards and regulations required for medical products in Australia. The system is intended to possess some or all of the following capabilities:
Achieve consistently accurate pressure range from 0-25 psi
Infusion flow rates of 0.1 to 50 ml/hr in 0.1 ml/hr increments and 50-500 ml/hr in 1.0 ml/hr increments.
Delivery system accuracy is required to be maintained at maximums of +/−6% (nominal) (ml).
Microprocessor controlled pump mechanism for precise electronic delivery of chemotherapy drugs.
Ability to deliver drug volumes of 1-999 ml in 1 ml increments.
Lifetime of all components no less than 6 years.
Overall weight of device (without drug fluid and batteries) less than 350 grams.
Auditory alarms distinct in varying pitch and frequency to minimise masking and be able to produce a sound-pressure level of at least 65 dB at 1 m.
Memory of the infusion pump should be able to record history reports of volumes infused and elapsed time for a minimum of 14 days.
Have a battery that lasts no less than 5 days in normal operation of 10 ml/hr.

Due to the nature of the continuous chemotherapy treatment, particularly continuous low dose treatment, the described IV fluid delivery system ought to be able to be taken with the patient at all times and be suitable for use in commonly foreseeable environments. Taking into account Australian conditions, the proposed demographic and the required control electronics, the following operating conditions are preferably satisfied. These operating criteria comply with Australian and International standards for medical electrical equipment.
Temperature: 0-45 degrees Celsius.
Humidity: 20-70 relative humidity.
Atmospheric pressure: 660-1060 bar.
Fluid ingress protection: IPX7.

Sensitivity in the design is given to design elements that allow the garment fit to be tailored to each patient and so that weight is generally evenly distributed across the garment to achieve patient comfort.

The manufacturing of non-garment parts of the system may predominately consist of plastic injection moulding and injection blow moulding elements. The housing structure that will be required to house certain components—pump mechanism, LED modules, flow regulator etc.—may require the use of such methods.

Material selection criteria for the main components of the pump device include the following:
Creep resistance—for dimensional stability of material to ensure dose accuracy.
Vibration and shock resistance—for durability in transport, dose accuracy.
High toughness—for impact resistance in operation, storage and transport.
Chemical resistance—for sterilisation and drug delivery, increased durability.
Bio-compatible—stable material to prevent or minimise allergic reactions in user for intravenous applications.
High strength to weight ratio—for durability, case of use and transport.

Component selection therefore focuses on the following options:
Precision components—Polybutylene Terephthalate (PTB), Polyoxymcthylene (POM).
Moving components—Polyamide (PA). POM.
Seals and grips—Thermoplastic Elastomer (TPE), rubber.
Harness and flexible/elastic elements—Polytetrafluroetylene such as GoreTex or NEXAR Polymers.
Non-critical components—PBT, PA, ABS, Hybrid Bio-plastics.
Chemotherapy drug reservoir—Polymethylmethacrylate (PMMA);
Fasteners—steel, adhesives.

Described embodiments may have one or more of the following characteristics:
Comfortable; non-intrusive to users' movement with a slim line fit, reduced product size and even weight distribution for being worn under clothing.
Easily assembled and programmed pump device, which automates actions or processes where possible.
Electronically driven pump mechanism for accuracy of dose administration.
Provides visual and auditory feedback for user inputs and safety alarms.
Continual vitals monitoring of user.

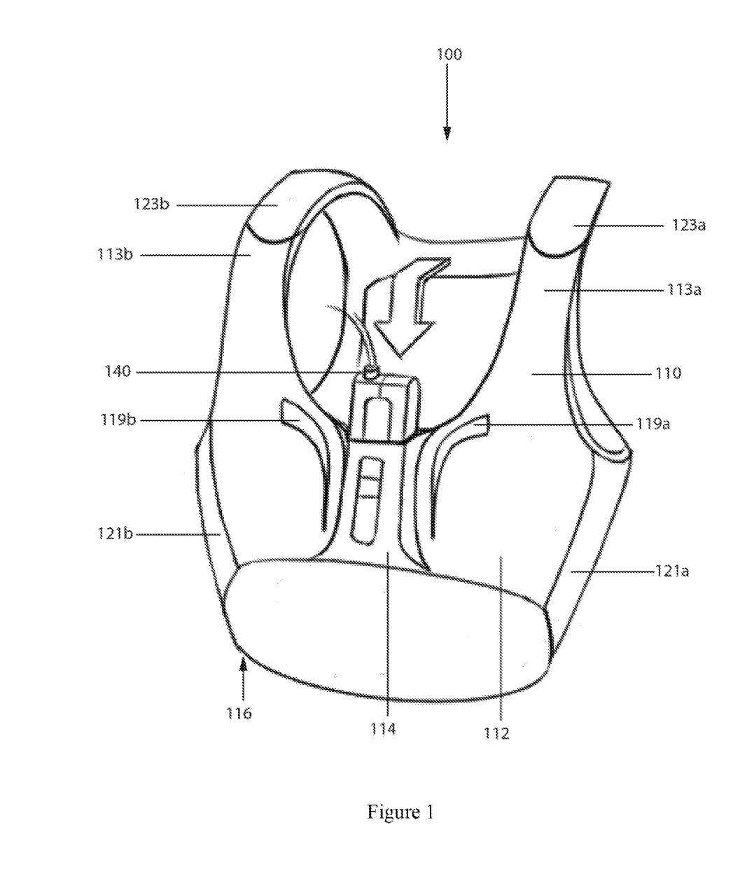

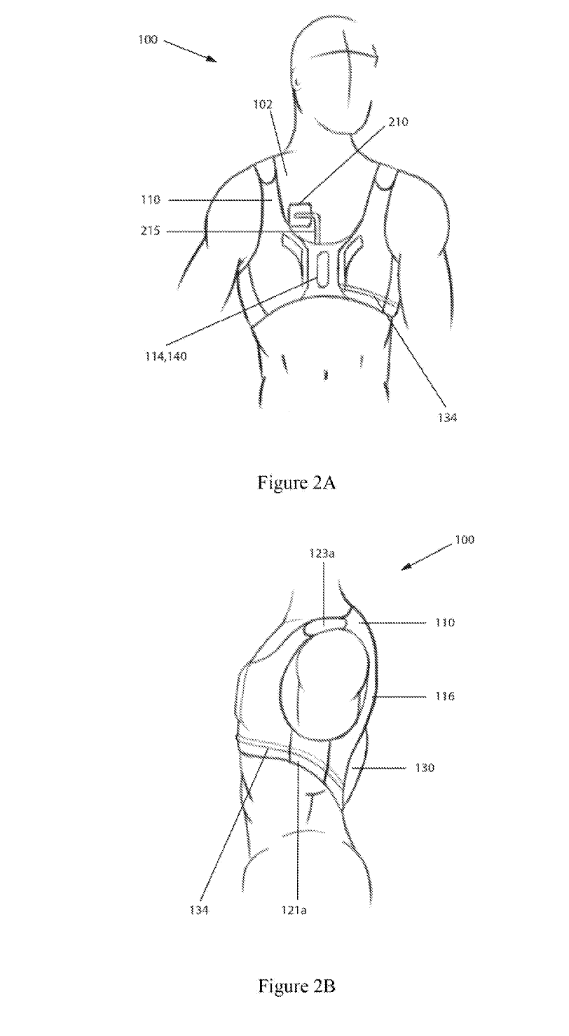

With reference firstly to FIGS. 1, 2A and 2B, a wearable system 100 for intravenous fluid delivery is described by way of example. System 100 comprises a wearable garment 110, which may resemble a vest in some embodiments, and a portable pump unit 140. The pump unit 140 acts as an infusion pump to deliver fluid from a fluid reservoir 130 to a delivery site 210 of a patient 102 via a fluid supply line 215. The wearable garment 110 has a pump unit support portion 114 to support and carry the pump unit 140 as it operates. In this example, support portion 114 is formed to resemble a pouch that snugly accommodates the pump unit 140 therein.

In example embodiments of the garment 110, most, or all of the garment 110 may be formed of one or more layers of a flexible and/or stretchable clothing material that can be worn next to the skin of the patient. The pump unit support portion 114 is thus preferably made of a suitable flexible material to accommodate receipt of pump unit 140 therein in a manner that would not readily allow it to accidently fall out or be withdrawn. The pump unit support portion 114 may have a window formed in a wall thereof in order to allow visual indicators or a display of the pump unit 140 to be inspected.

The garment 110 has a front side 112 and an opposite back side 116 which are sized and/or configured to overlie the anterior and posterior regions of a human upper body. The front section 112 may generally overlie the thorax and may in some versions be configured to be worn by a male and in other versions to be worn by a female. In configurations of garment 110 to be worn by a female, the front section 112 may be configured to allow for comfortable accommodation of the breasts and may function at least partly as a brassiere. As would be appreciated, garment 110 when formed of flexible or stretchable clothing material will be tight fitting and wearable under a patient's other garments.

The front section 112 is coupled to the back section 116 via side portions 121a, 121b on the left and the right sides of the front portion 112. Front portion 112 further comprises straps 113a, 113b that extend upward and over the shoulder to join with the back section 116. Straps 113a, 113b may have adjustable strap lengths, which can be manually adjusted by strap couplings 123a, 123b respectfully. In some embodiments, the front section 112 may have vent sections 119a, 119b on either lateral side of the pump unit support portion 114. These vent sections 119a, 119b may additionally or alternatively be formed of a material that has greater stretching capability than the surrounding material of the front section 112, for example to allow for anatomic variation among patients.

In the version of system 100 shown on FIGS. 1, 2A and 2B, a fluid reservoir support portion 130 is located on the back section 116. The fluid reservoir to be received in the fluid reservoir support portion 130 will be formed as an enclosed bladder of fluid with an outlet that can be coupled to system 100 as part of a set-up process performed by medical or other assistive personnel. The same system 100 may be used to deliver fluids from a number of separate fluid reservoirs. It is not envisaged that the fluid reservoir will be reused, so a new fluid bladder will be need for each treatment.

The fluid reservoir only forms part of the system 100 when it is received in the fluid reservoir support portion 130 and is fluidly coupled to an inlet conduit 134. In order to convey fluid from the fluid reservoir (when received in and supported by the support portion 130) to the delivery site 210, an inlet conduit 134, in the form of a small flexible tube, is carried by the garment 110 and fluidly connects the fluid reservoir (when present and coupled thereto) with an inlet of the pump unit 140. The inlet conduit 134 may extend from the back 16 of garment 110 to the front 112 by either the left side or right side of the garment and is preferably concealed and sandwiched in between layers of material of the garment 110.

The fluid reservoir support portion 130 may be positioned so as to have the fluid reservoir, when present, overlie at least pan of the spine of the patient and is preferably centrally aligned with respect to the spine. The fluid reservoir support portion 130 may hold the fluid reservoir, when present, close to the skin of the patient's back (although it may be separated therefrom by at least a thin layer of material) in a region around the thoracic and/or lumbar spine. In this way, the temperature of the fluid being delivered into the patient's body via delivery site 210 can be kept relatively close to the patient's body temperature, since the fluid reservoir will be close to the skin.

The wearable IV drug delivery system 100 of FIGS. 1, 2A and 2B may vary from the form depicted and described in relation to the drawings. For example, the location of the fluid reservoir support portion 130 may be on the front of the garment instead of the back, the central location of the pump unit support portion 114 may be repositioned, for example toward the side or the back of the garment and the vest like appearance may be modified to reduce or minimise the amount of clothing material that forms the garment 110.

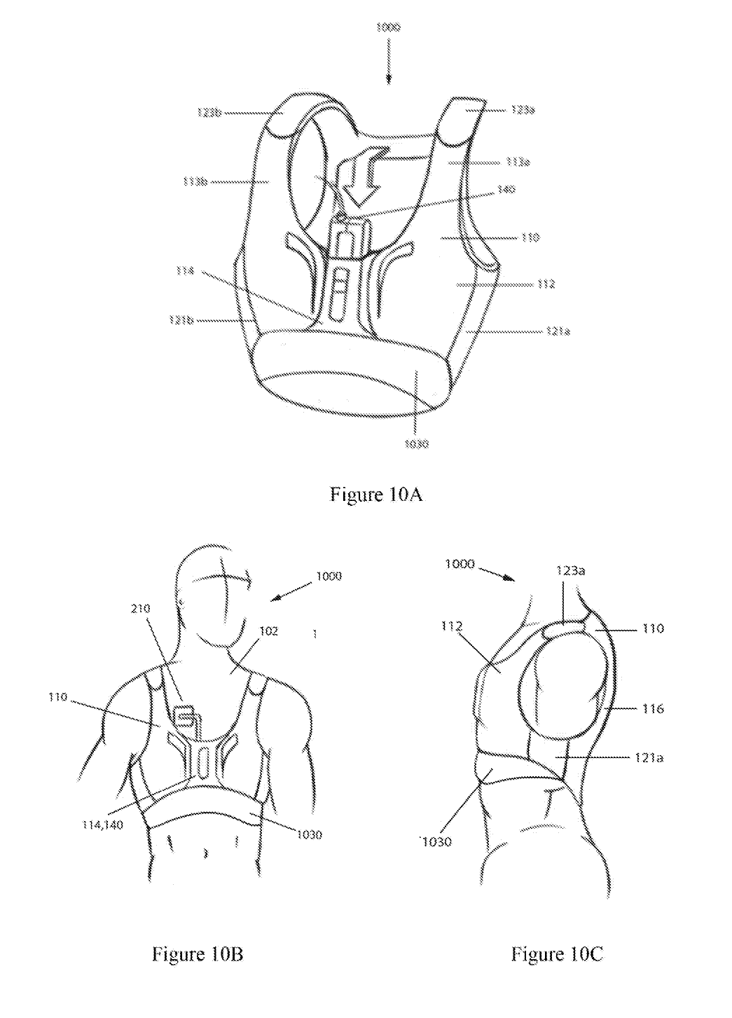

FIGS. 10A, 10B and 10C illustrate another example IV fluid delivery system 1000 that is substantially the same as system 100, but for the position of a fluid delivery support portion 1030 that is positioned on the front 112 of the garment 110, rather than the back 116. The fluid reservoir support portion 1030 may be positioned immediately below the pump unit support portion 114 to minimise a distance that the fluid needs to travel from the fluid reservoir to the pump unit 140. If the fluid reservoir is to be received in the fluid reservoir support portion 1030 on the front of the garment 110 then it may be positioned (when present) within the support portion 1030 to generally extend across at least part of the abdomen and/or thorax of the patient. Preferably, the fluid reservoir is sized and shaped to be received generally symmetrically with respect to a centre line of the patient (ie with respect to a vertical line through the umbilicus). In IV fluid delivery system 1000, the inlet fluid conduit 134 is not shown, although it may be provided as a very short length of conduit extending between the fluid reservoir (when present) and the pump unit 140. Other than the differences noted above, the description provided above in relation to IV fluid delivery system 100 also applies to the IV fluid delivery system 1000.

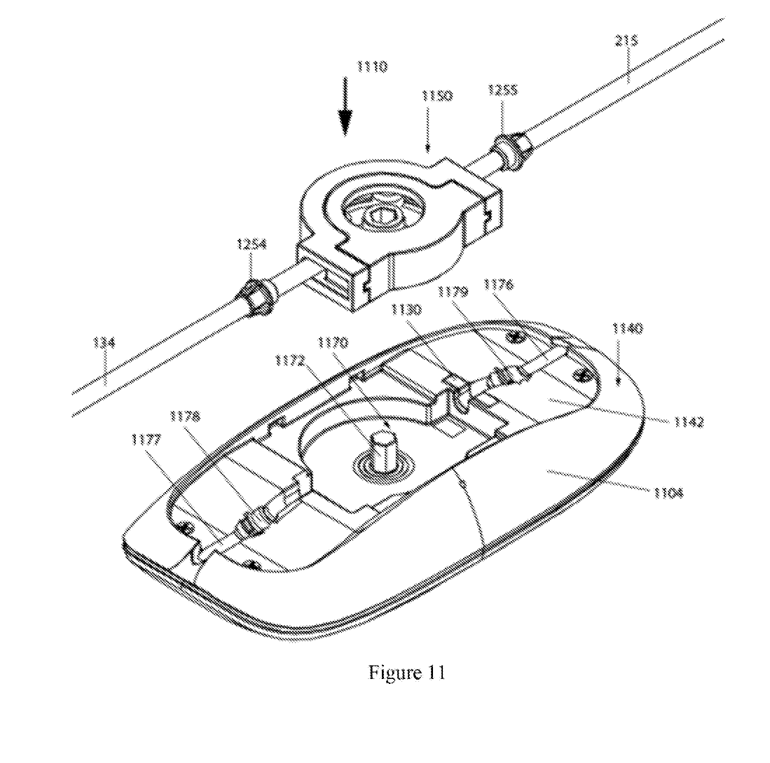

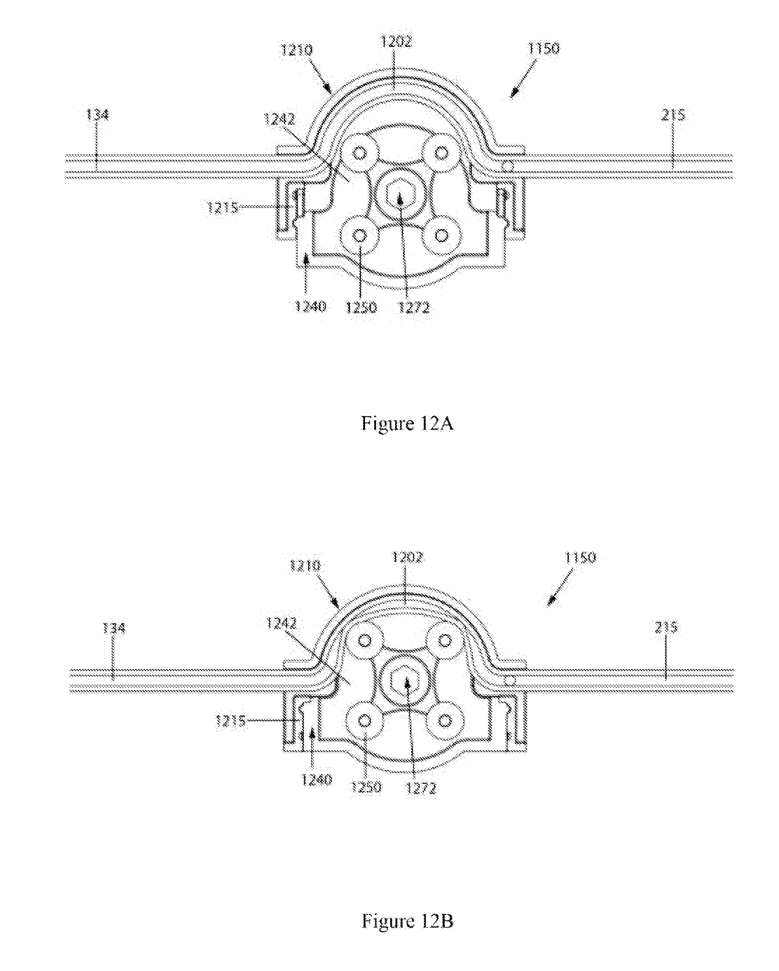

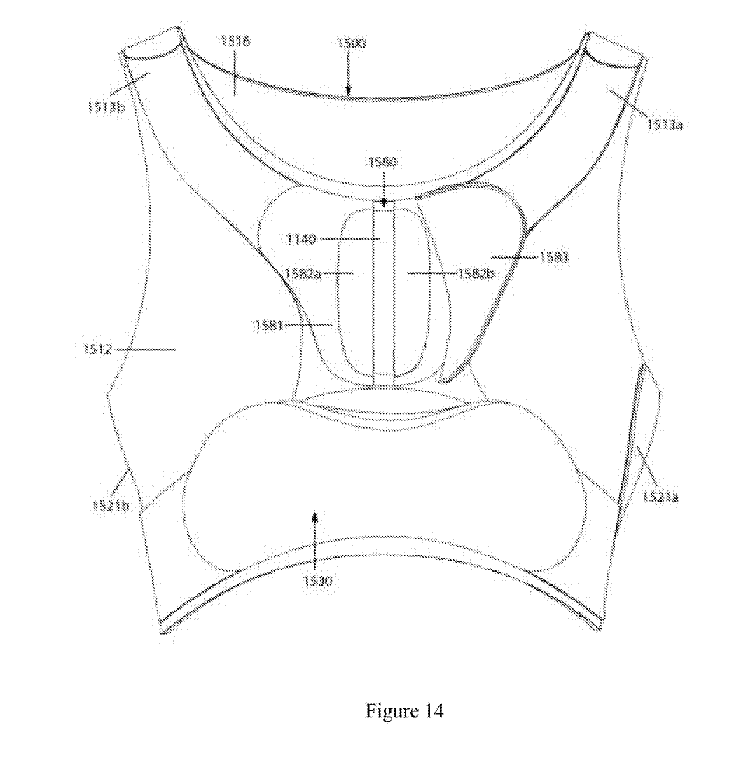

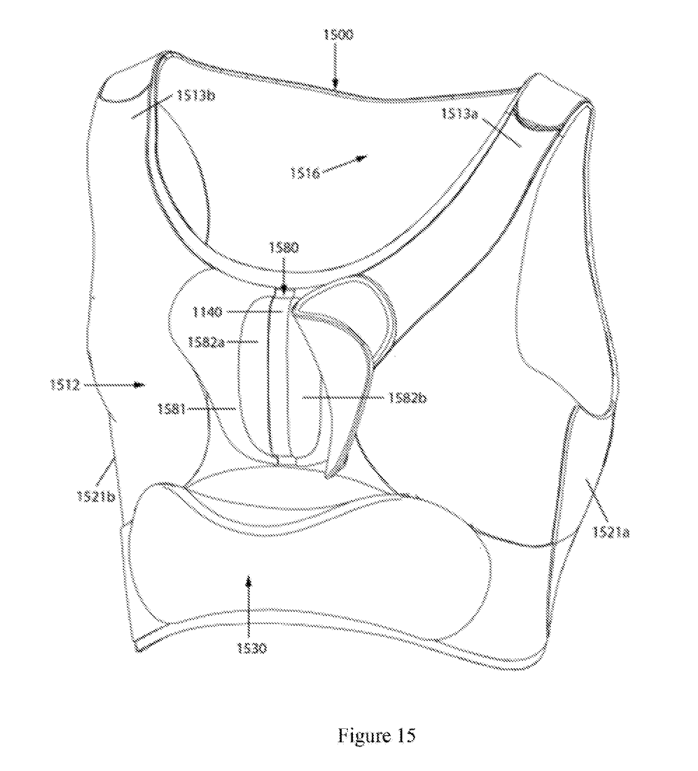

Referring now to FIGS. 14 and 15, there are shown front and perspective views of wearable garment or vest 1500 according to another illustrative embodiment. Garment 1500 includes a pump unit support portion 1580 for supporting a pump unit 1140 of the type illustrated in FIG. 11 and a fluid reservoir support portion 1530. Similar to the example garment 110 illustrated in FIGS. 10A-10C, the fluid reservoir support portion 1530 in this embodiment is located at the front of garment 1500 below pump unit support portion 1580 and extends laterally to be positioned over the abdomen of the patient.

Garment 1500 is configured as a vest and includes a front section 1512 and an opposite back section 1516 which are shaped and sized to closely fit the respective anterior and posterior regions of a human upper body so that garment 1500 may be worn under other garments. The front section 1512 is joined or coupled to back section 1516 by side portions 1521a, 1521b that extend under the arms of a patient wearing garment 1500. In addition, garment 1500 includes shoulder straps 1513a and 1513b that each extends over respective shoulders to be joined to the back section 1516.

In this example, pump unit support portion 1580 includes a centrally disposed pump unit receiving region 1581 and a pair of opposed pockets 1582a. 1582b which open to receive and hold the pump unit 1140 in place in receiving region 1581. Pump unit support portion 1580 further includes a cover 1583 in the form of a securement flap that extends from one side of the pump unit receiving region 1581 to overlie the pump unit 1140 and which attaches to the other side of the pump unit receiving region 1581 by conventional means such as by the use of Velcro tabs or equivalent. In this manner, pump unit is securely retained in both vertical and horizontal directions and an operator is able to view any warning or status indicators on the pump unit 1140 by opening securement flap 1583 and viewing pump unit 1140 through the gap between pockets 1582a and 1582b.

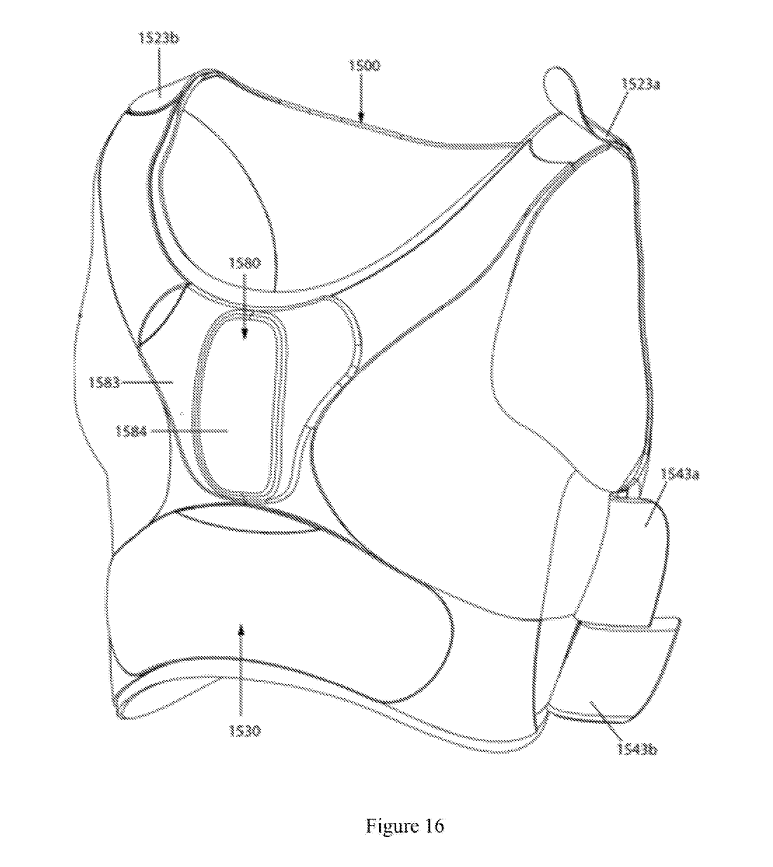

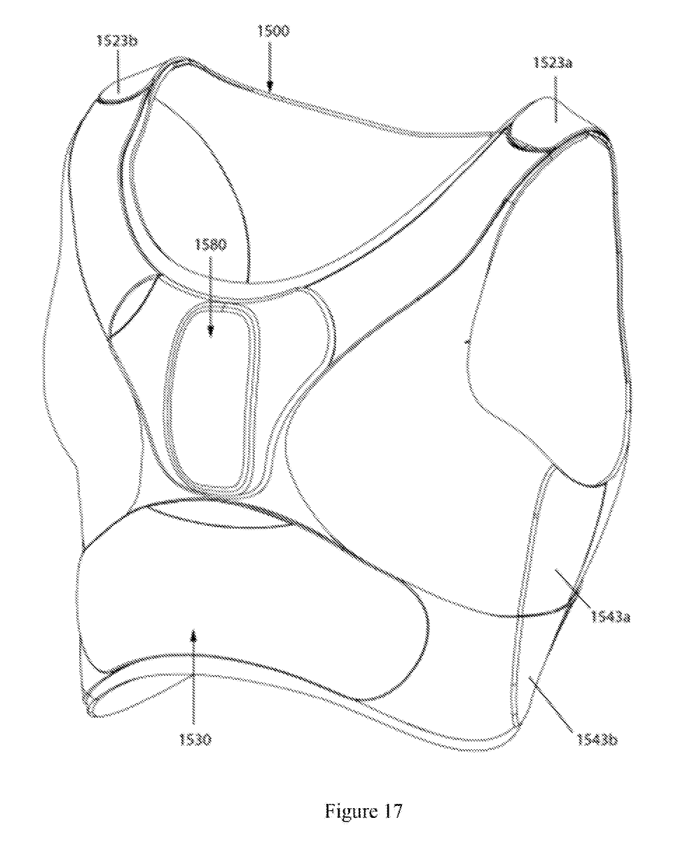

Referring now to FIGS. 16 and 17, there is shown the securement flap 1583 in a closed position. As can be seen in these views, securement flap 1583 also includes a complementary front receiving region 1584 formed in the material of securement flap 1584 that is shaped and sized to conform to the front surface of pump unit 1140 again functioning to comfortably locate and retain pump unit 1140.

As shown in FIG. 16, the sizing of garment 1500 may be adjusted by the use of side coupling straps 1543a, 1543b as well as the shoulder coupling straps 1523a, 1523b similar to those described previously (eg, see FIG. 8). Side coupling straps 1543a, 1543b allow even further adjustment of garment 1500 depending on the body shape of the patient. As would be appreciated, side coupling straps 1543a, 1543b may be deployed on both sides of garment 1500 to provide even further adjustability.

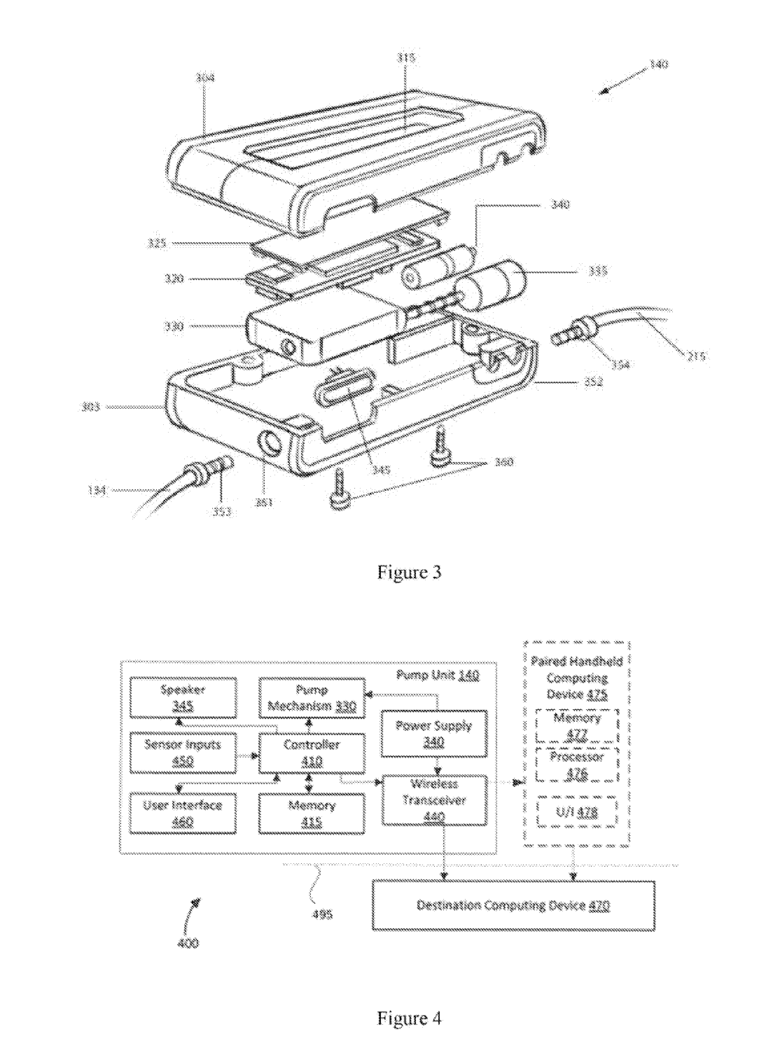

Referring now to FIG. 3, pump unit 140 is shown and described in further detail. Pump unit 140 comprises a housing having upper and lower casing parts 304, 303, respectively. The casing parts 303, 304, when assembled and fixed to each other, house and enclose a fluid tight environment in which a pump mechanism 330 is disposed, together with a drive motor 335 configured to operate the pump mechanism 330. Also enclosed within the housing is a power source 340, for example in the form of a rechargeable battery to provide power to the pump unit 140. A control board 320 is also comprised in the housing and serves to operate the drive motor 335 while drawing power from power source 340 and controlling the supply of power from power source 340 to drive mechanism 335. Control board 320 may drive a display panel 325 to provide a user interface display viewable through a window 315 formed in the upper casing part 304. A speaker 345 may also be housed in the housing and arranged to emit audible alarm signals in response to control signals from the control board 320. Fasteners 360, together with optional snap fittings and cooperating structures of the housing, may be used to fasten the upper and lower casing parts 304 and 303 together.

The lower casing part 303 may comprise an inlet port 351 to receive a fluid coupling jack 353 at one end of the fluid inlet conduit 134. Similarly, at an opposite end of the lower casing part 303, an outlet port 352 may be formed to receive an outlet coupling jack 354 in communication with the fluid supply conduit 215. The pump mechanism 330 is arranged to convey fluid from the inlet port 351 to the outlet port 352 in a measured manner according to control parameters programmed into the control board 320 (at least some of which may be user-selectable.) In some embodiments, such as are exemplified by pump unit 1140 in FIG. 11, the pump unit may be configured so that the fluid does not actually enter and exit the housing of the pump unit but is nevertheless conveyed toward the delivery site 210.

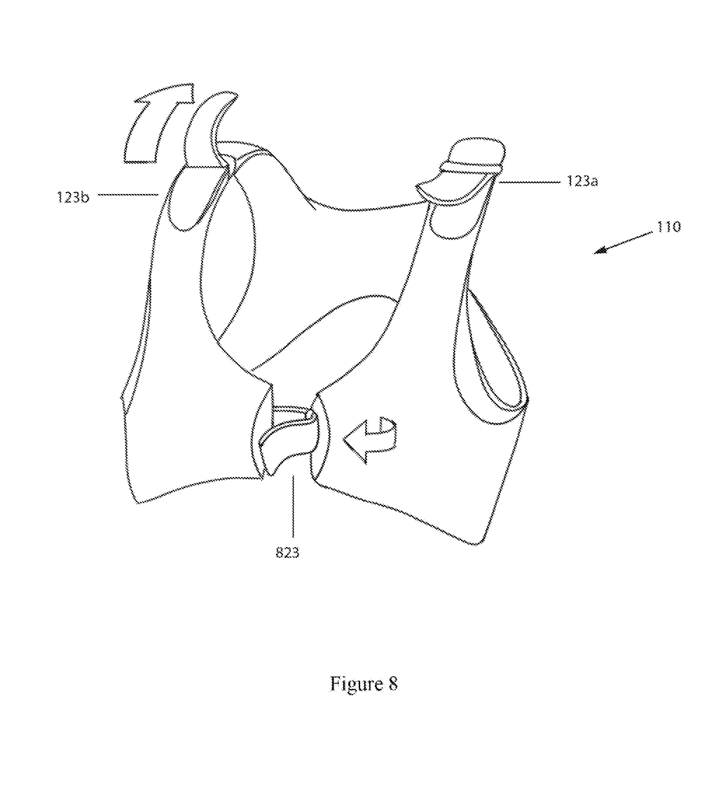

Although not shown, pump unit 140 may have a communication port, such as a universal serial bus (USB) port or other communication interface, to allow the pump unit 140 to be communicatively coupled to another device such as the computing device 710 (FIG. 7) or a dock 750 having a program interface 760 (FIG. 7).

Referring now to FIG. 4, a fluid delivery monitoring system 400 is described. The fluid delivery monitoring system 400) may be used in concert with the IV fluid delivery system 100 so that when the pump unit 140 is operating to pump fluid into delivery site 210, the pump unit 140 also provides a monitoring function and communicates data of monitored sensor outputs to a destination computing device 470 for this purpose.

As shown in block diagram form in FIG. 4, the pump unit 140 has a controller 410 and memory 415 (e.g. carried on control board 320) and additionally includes a wireless transmitter or transceiver 440. The memory 415 stores executable program code and configuration parameters for operation of pump unit 140, including controlling operation of the pump mechanism 330, communicating to other devices via wireless transceiver 440 and presenting output and receiving input (if applicable) via user interface 416, which may include display 325 and/or other indicators. Additionally, the pump unit 140 may receive sensor inputs 450 from at least one sensor on or carried by garment 110 in a respective sensor carrying portion or elsewhere on the body of the patient. Such sensor inputs 450 may indicate at least one biological condition of the wearer of the system 100 or 1000, such as body temperature, heart rate, pulse, respiratory rate, electrocardiogram signals, respiratory noise, blood pressure and/or blood oxygen saturation, for example.

In some embodiments, the system 400 includes a handheld computing device 475, such as a smart phone, that is paired (ie communicatively coupled) to the pump unit 140 via the wireless transceiver 440. This handheld computing device 475 may have normal smart phone functions to allow the execution (by a processor 476 executing program code stored in a memory 477) of an application that interfaces with the pump unit and provides a user interface 478 for the patient to interact with. This user interface 478 may show sensor readings and trending over time for the sensor readings, as well as time elapsed or remaining for the treatment, how much fluid has been infused and optionally at what rate.

Additionally, the handheld computing device 475 may provide added detail, such as an alarm description, to a patient about any alarm indication or notification transmitted from the pump unit 140. Further, the handheld computing device may be configured to act as the smart communication and user interface for the pump unit 140 by automatically notifying (by email, text message or other electronic notification) one or more designated contacts, such as doctors, relatives or other concerned persons in the event that an alarm condition is triggered. This notification may vary, depending on the nature of the alarm condition. For example, a low body temperature alarm condition may be notified to the patient's doctor, while a sensor or pump fault alarm condition may be notified to a nurse and/or relative to take corrective action. In any case, the electronic notification is sent over a network interface 495 (which may include a GPRS network, the Internet, a local area or WiFi network or a combination of such networks) to a destination computing device 470 belonging to or associated with the designated contact. Additionally, the handheld computing device may have a user-selectable option to notify any of the designated contacts if the patient is feeling particularly unwell or has concerns about proper functioning of the treatment and monitoring system.

In some embodiments, the handheld computing device 475 may not be present and the pump unit 140 may determine relevant alarm conditions itself and transmit suitable notifications to the designated contacts (at respective destination computing devices 470) over the network interface 495. However, if present, the handheld computing device 475 may act as a communications gateway for the pump unit 140, so that the battery of the pump unit is not unduly drained.

The system 400 may comprise a number of sensors carried by the garment 110 or another part of the patient's body and in communication with the pump unit 140. Such sensors may be electrically coupled to a suitable input jack (not shown) on the pump unit 140 or may be configured to wirelessly communicate with the wireless transmitter 440 a pump unit 140 using a low power short range personal area network (PAN) or wireless PAN protocol. The personal area network may include a wireless body area network (WBAN), sometimes referred to as a body sensor network (BSN). Although it is desired that at least one temperature sensor be carried by the garment 110 and positioned so that it contacts the skin or is at least closely adjacent to the skin, and provides its output to the controller 410, other sensors 455 providing signals indicative of relevant biological conditions may be worn by the patient 102 in other ways. For example, a blood pressure sensor may be worn elsewhere on the body or a blood oxygen saturation sensor may be worn on an earlobe, for example.

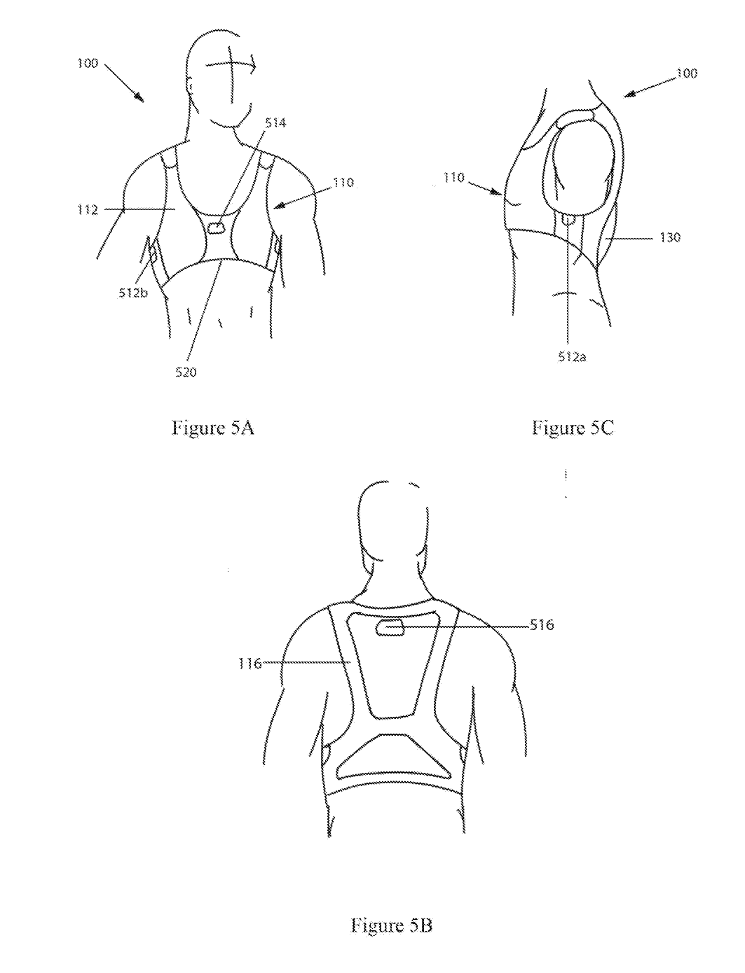

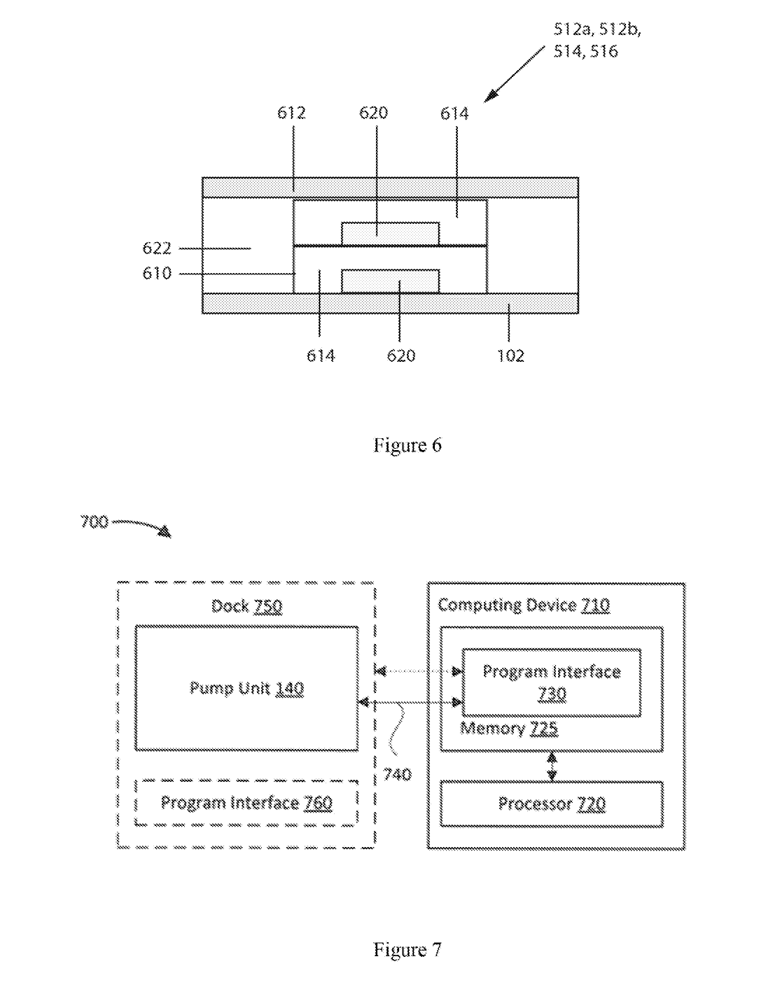

Referring also now to FIGS. 5A, 5B, 5C and 6, example positions of sensors carried by the garment 110 in respective sensor carrying portions are described. Garment 110 may comprise multiple temperature sensors, such as temperature sensors 512a and 512b at sensor carrying portions located at either side of the rib cage under the arm in the side sections 121a, 121b of the garment 110. Temperature sensor 514 is located in a sensor carrying portion in a front central portion of garment 110 on front side 112 at the sternum. Another temperature sensor 516 may be positioned towards the top of the back section 116 in a corresponding sensor carrying portion to generally overlie a top of the thoracic spine or upper thoracic region. Core body temperature sensors may be positioned in locations other than those locations shown in the Figures and include locating a tympanic temperature sensor to sense the temperature of the inner ear. Pulse/blood oxygen sensor readings can be taken from the earlobe and finger, blood pressure sensor readings from the arm, and ECG, heart-rate and respiratory monitoring from the front, back and sides of the torso. Sensors taking these readings may each be coupled to the pump unit 140 (providing sensor inputs 450) by small wires or wirelessly.

FIG. 6 illustrates an example arrangement of the temperature sensors 512a, 512b, 514 and 516. Such temperature sensors may comprise two thermistor sensors layered vertically (one on top of the other) adjacent to the skin surface. This allows the lower thermistor to measure correct contact skin temperature while the secondary temperature measures radiant body temperature. These two values can be calibrated to calculate inner core body temperature with an accuracy of around 0.1 Degrees Celsius. In FIG. 6, the first and second thermistors are indicated by 610 and 612 respectively. Each thermistor 610, 612 is at least partly encased by an insulation material 614, although the first thermistor 610 is exposed to the skin of patient 102. The thermistors 610, 612 each have a resistive element 620. The thermistors 610, 612 may be encased in a silicone carrier 622 and received by or within the sensor carrying portion of the garment which in one example may be a small pocket within the garment 110 that the sensor may be sewn into.

The garment 110 may also carry at least one further sensor 520 to sense heart rate, ECG (i.e. cardiac noise) and respiratory functions, such as respiratory noise and breathing rate. Such additional sensors 520 may be positioned at a lower central part of the front side 112 of the garment 110, for example as shown in FIG. 5A or at another suitable location.

FIG. 13 illustrates in block diagram form how the sensors described above may form nodes of a PAN or BAN within the monitoring system 400, along with the pump unit and optionally the paired handheld computing device 475. The PAN or BAN may include a central repeater node to facilitate communication between all of the PAN or BAN network nodes (ie, the pump unit 140, the sensors 512a, 512b, 514, 516, 520, 455 and the handheld computing device 475) or may facilitate communication directly between the handheld computing device 475 and sensors in addition to or instead of communicating with the pump unit 140.

Referring now to FIG. 7, a pump unit configuration system 700 is shown. The pump unit 140 may be communicatively coupled to a computing device 710, for example via a wireless connection or a wired cable 740, such as a USB cable. This coupling allows the memory 415 of pump unit 140 to be programmed with suitable control parameters and other code necessary for the operation of the pump unit 140 via a program interface 730. The program interface 730 may be executed by a processor 720 of the computing device 710 that generates the program interface 730 based on software code stored in a memory 725 of the computing device 710. The program interface 730 preferably provides a user friendly interface to readily allow nurses or other medical staff to appropriately configure the pump unit 140 prior to placing it in the pump unit support portion 114 and coupling it to fluid supply line 215.

In alternative embodiments of configuration system 700, the pump unit 140 may be received in a suitable dock 750 that has a program interface 760 hosted by suitable hardware and/or software incorporated within or accessible to the dock 750. If the pump unit 140 is coupled to the dock 750 for configuration, then the pump unit 140 may have a suitable hardware docking interface to allow data transfer and/or battery charging.

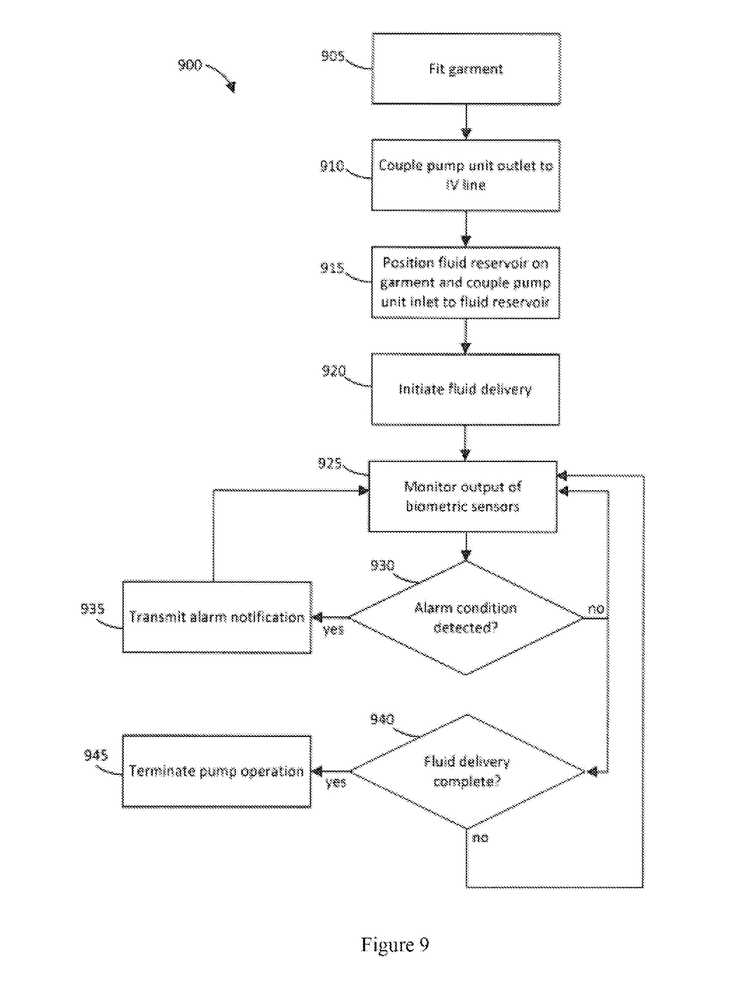

Referring now to FIG. 9, a method of intravenous fluid delivery 900 is described. At 905, the garment 110 is fitted to the patient 102 to be snug and to lie adjacent to the skin. The garment 110 should be arranged to be tight fitting and hug the upper body of the patient so that is can be easily worn under other clothing, yet not so tight as to provide discomfort to the patient 102. In fitting the garment 110 at 905, the length of the shoulder straps 113a, 113b can be adjusted by adjustable couplings 123a, 123b, as shown in FIG. 8. Further, the garment 110 may have a means for adjusting the tightness of fit around the circumference of the thorax, for example including an adjustable strap 823 on the front 112 (which may be hidden under one or more layers of the garment 110).

Once the garment 110 has been fitted to the patient 102, the pump unit 140 is coupled at 910 to provide fluid to outlet conduit 215. This method 900 assumes that the delivery site 210 has already been suitably secured by medical personnel and the pump unit 140 has already been configured appropriately for delivery of a set amount of fluid over a set time.

At 915, the fluid reservoir is positioned within the fluid reservoir support portion 130 or 1030 and is coupled to the pump unit 140 to provide fluid for communication by the pump unit 140 to the delivery site 210. At 920, the pump unit 140 is activated to initiate fluid delivery in the manner configured and the pump unit 140 proceeds to pump fluid to the delivery site 210 until the fluid reservoir is exhausted or until a specified period expires.

At 925, the pump unit 140 or the paired handheld computing device 475 (which in some embodiments may receive the sensor outputs instead of or in addition to the pump unit 140) monitors the output of the various biometric sensors coupled to the garment 110 or otherwise forming part of the personal area network or body area network. If the pump unit 140 or the paired handheld computing device 475 determines at 930 that an alarm condition has been detected (based on predetermined alarm conditions relating body temperature variation, heart rate variation, respiratory noise level, for example), then at 935 an alarm notification is transmitted to the destination computing device 470, so that suitable medical personnel can take heed of the alarm condition and initiate appropriate action. Depending on the particular alarm conditioned detected, the pump mechanism of the pump unit 140 may cease operation or may continue operation. Irrespective of the continued operation (or not) of the pump mechanism of the pump unit 140, the pump unit 140 or the paired handheld computing device 475 continues to monitor the output of the biometric sensors at 925, in case further alarm conditions are detected and are required to be notified.

Where no alarm conditions are detected at 930, then the pump unit 140 continues to perform its fluid delivery function until it determines at 940 that the fluid delivery is complete or another predetermined condition (such as elapsed time) has been satisfied, and the pump unit 140 then terminates operation of the pump mechanism 330. During operation of the pump unit 140, the controller 410 may transmit frequent update messages (ie at an interval that can be set to range from every second to every minute or more) to the handheld computing device 475 (if it is present), so that the patient can track the status of the treatment.

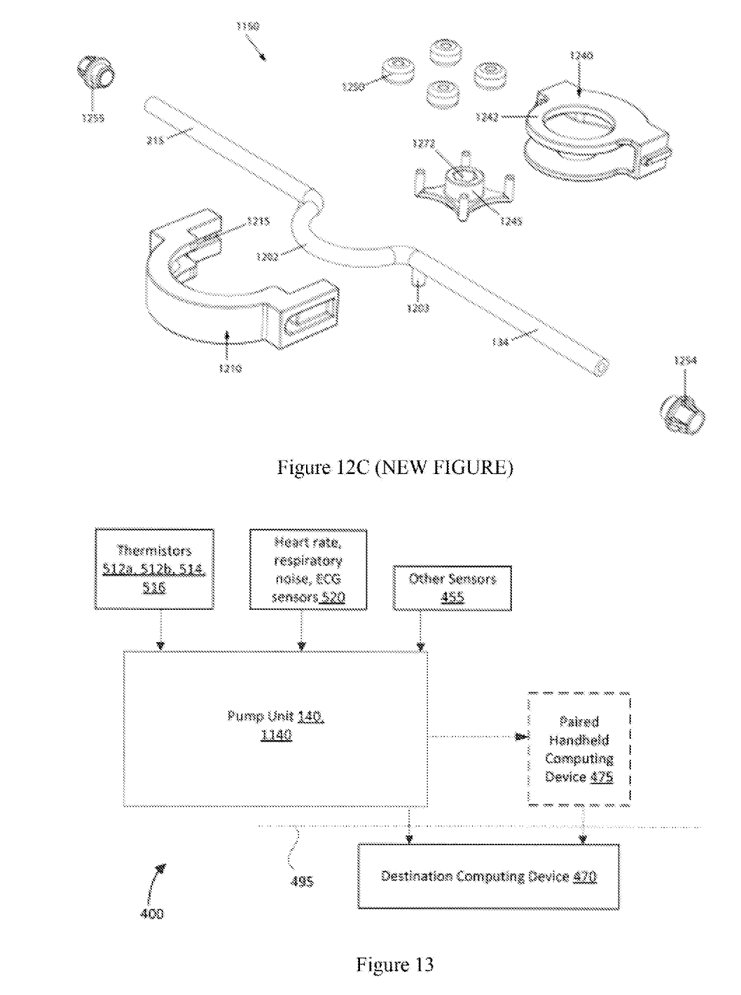

Referring now to FIGS. 11, 12A, 12B and 12C, an example peristaltic pump mechanism is shown. Some embodiments may employ an example pump unit 1140 that uses a peristaltic pump mechanism 1150 to pump fluid to the delivery site 210. The peristaltic pump mechanism 1150 comprises a first component in the form of frame 1210 that is removably attached by frictional engagement by clipping or similar operation to a section 1202 of fluid supply conduit that is continuous with inlet conduit 134 and outlet conduit 215. The frame 1210 and the attached fluid supply conduit 1202 which now traces an arcuate path is arranged to receive a complementary second component in the form of a peristaltic pump cassette 1240. Pump cassette 1240 comprises a rotating member or rotor 1245 to which are coupled a plurality of rollers 1250 that engage peristaltically with the conduit section 1202 by travelling along the defined arcuate path. This causes fluid to move along the conduit section 1202 as the rotor 1245 rotates under the action of a drive shaft 1172 driven by an internal motor (not shown) of the pump unit 1140. The rotor 1245 is carried by a cassette frame 1242 in a manner that allows rotation of the rotor 1245 with respect to the frame 1242. The rotor 1245 has a central hexagonal shaped aperture 1272 to receive the drive shaft 1172 in mating engagement so that the rotation of the drive shaft 1172 causes corresponding rotation of the rotor 1245.

The frame 1210 comprises a cassette receiving portion 1215 and a conduit receiving portion 1212. When the peristaltic pump cassette 1240 is matingly received in the frame 1210, the combination of the frame 1210 and cassette 1240 provides a peristaltic pump mechanism 1150.

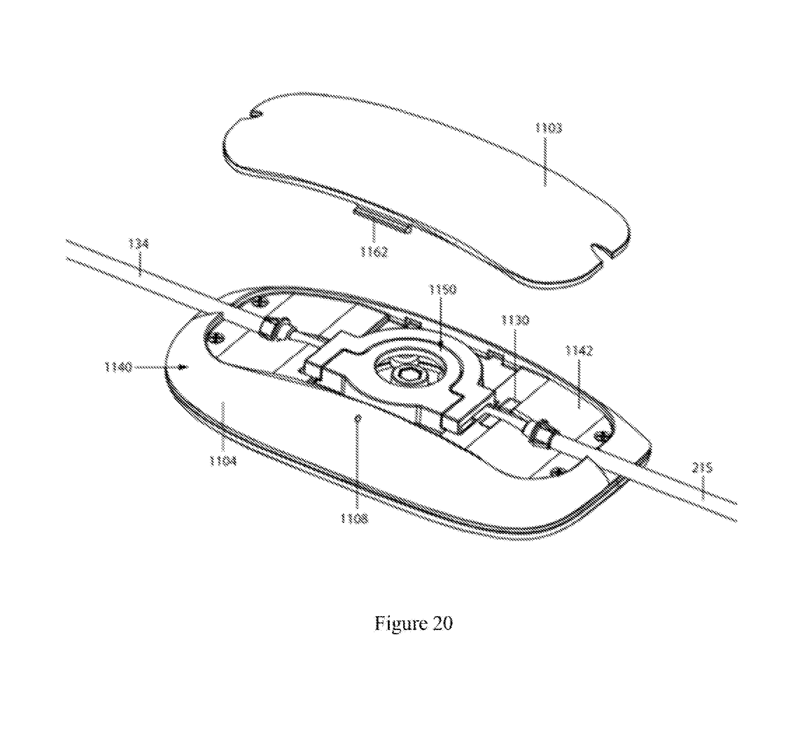

As part of setting up the IV fluid delivery system 100 or 1000, the pump unit 1140 may be used in place of the pump unit 140 and the peristaltic pump mechanism 150 may be separately attached or coupled to the fluid supply line, which may be a single continuous flexible line as shown. The pump mechanism 1150 may then be positioned in the correspondingly shaped recess 1170 formed in a back section or pump mechanism receiving portion 1142 of the pump unit housing 1104. When the pump mechanism 1150 is inserted into the recess 1170 in the manner indicated by arrow 1110, the drive shaft 1172 is received within the corresponding aperture 1272 in the pump mechanism 1150 and the frame and cassette 1210, 1240 are snugly bounded by a lower surface and side walls defining the recess 1170. Additionally, the inlet fluid conduit 134 is received within a channel 1177 that has at least some retention structure 1178 tending to retain the conduit 134 in the channel 1177 once it has been pressed into the channel 1177 by mating with collar member 1254. Similarly, the outlet conduit 215 is received within a channel 1176 formed in the back section 1142 having a corresponding retention structure 1179 that mates with associated collar member 1255 (see also FIG. 20). When the pump mechanism 1150 is so received in the back section 1142 of the pump unit 1140, it may be retained in place by snap fitting engagement structures and preferably lies flush with a surface of the back section 1142. In this manner, the peristaltic pump mechanism is conveniently removably coupled to the pump drive arrangement or pump unit 1140 to allow attachment of the fluid supply conduit to the peristaltic pump mechanism as required.

The pump unit 1140 may have the same componentry and structure as pump unit 140, except that it uses a different pump mechanism and does not require fluid coupling jacks 351, 352 in the lower casing 303, since the inlet and outlet conduits 134, 215 are continuous with the conduit section 1202 that extends through the pump mechanism 1150. As would be appreciated, pump unit 1140 need not be fluid tight as the fluid supply conduit is continuous as it extends through the pump unit 1140.

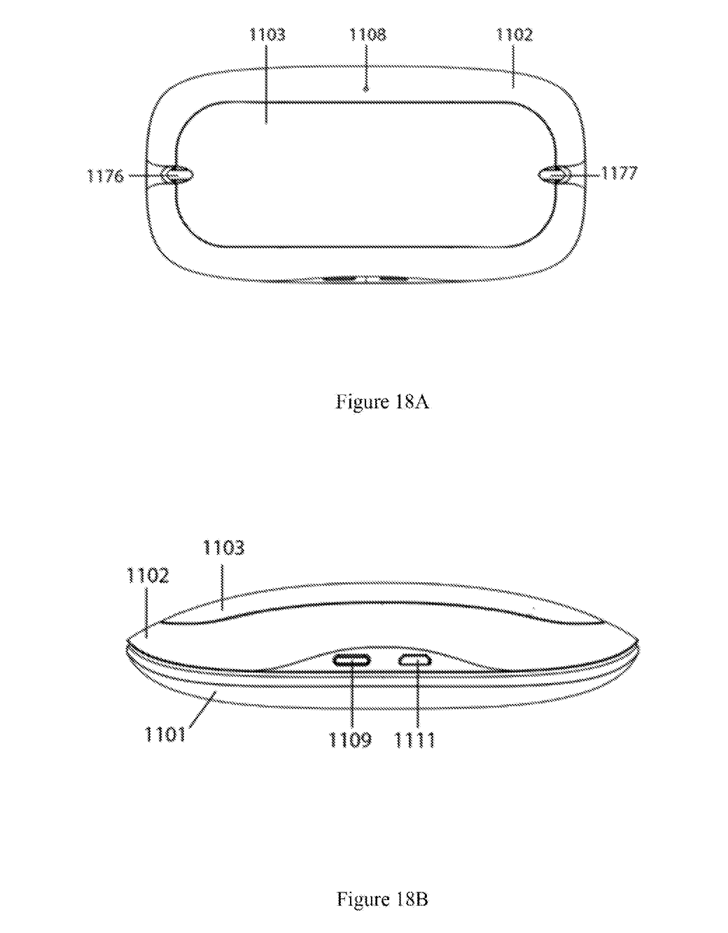

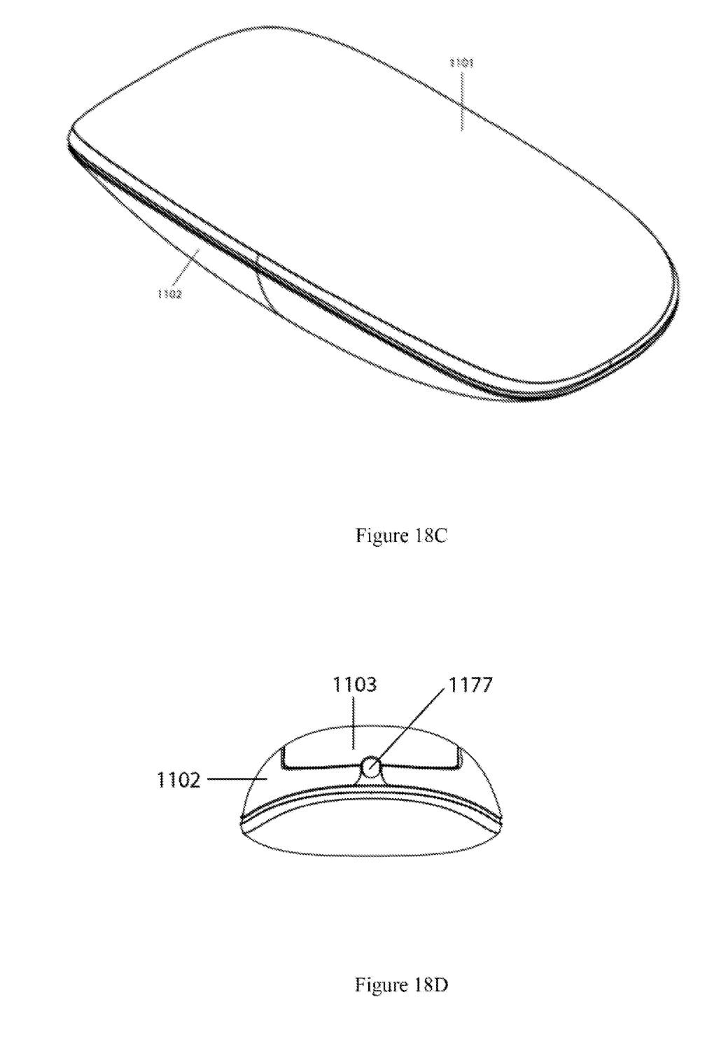

Referring now to FIGS. 18A-18D, there are shown various views of pump unit 1140 illustrated in FIG. 11. Pump unit 1140 includes a pump unit housing 1104 incorporating a housing front section 1101, and rear section 1102 and a pump mechanism cover 1103 which in use covers the pump receiving portion 1142 of housing 1104. Pump unit 1140 includes a power on/off switch 1109 and a micro-USB port 1111 for data communications. As best seen in FIG. 18A, located on opposed sides of housing 1104 are the respective apertures for inlet channel 1177 and outlet channel 1176 through which the continuous fluid supply conduit extends as described previously.

In this example, the combination of pump housing 1104 and pump mechanism cover 1103 together provides a contoured geometry adapted for ergonomic fitting to the front thoracic region of a patient allowing it to be readily seated in garment 1500 as shown in FIGS. 14 to 17. As would be appreciated, this geometry further assists in garment 1500 being wearable under further outer garments.

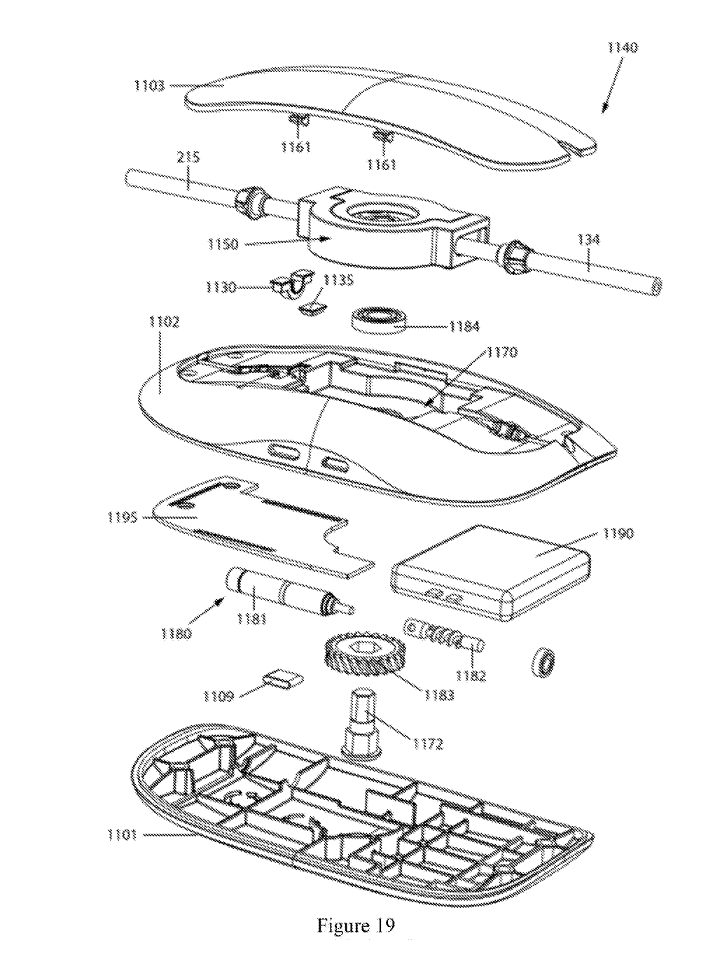

Referring now to FIG. 19, there is shown an exploded view of pump unit 1140 including pump mechanism 1150. Located within pump unit housing 1104 is the pump drive mechanism 1180, and battery power supply 1190 and control board 1195. Pump drive mechanism 1180 comprises an electric motor 1181 that drives a worm gear arrangement comprising a worm member 1182 which on rotation causes worm wheel 1183 to be rotated. This in turn drives hexagonal profile shaft 1172 extending through bearing 1184 and into recess 1170 to drive pump mechanism 1150 as has been described previously.

Control board 1195 implements the controller functionality allowing for set up of the pump unit and monitoring of sensor inputs as well as the provision of status indicators. For this embodiment, there is single visual status indicator attached to control board 1195 (not shown) that emits different coloured indicator signals through a thin walled portion of housing front section 1101. In this example, interface with the controller is by micro-USB port 1111 which also can function to charge the battery power supply as well as receive sensor inputs. In other embodiments, and as has been previously described, pump unit 1140 may incorporate a wireless transceiver to interface to other computing devices and/or to receive sensor inputs from other sensors carried by garment 1500. Optionally, pump unit 1140 may include an audible indicator such as a PCB mounted piezo electric speaker.

Pump unit 1140 includes an air-in-line sensor 1130 which in this example is an ultrasonic ceramic sensor having a semi-circular design to receive the fluid supply conduit. Sensor 1130 is interfaced with control board 1195 which monitors its status and generates a warning indicator if air is detected in the fluid supply conduit. Pump unit 1140 further includes a ceramic pressure sensor 1135 interfaced to control board 1195 which on assembly is located below the downwardly extending portion 1203 of the fluid supply conduit (as best seen in FIG. 12c) to measure the pressure of the fluid flowing through fluid supply conduit. Again, a warning indication may be generated if the fluid supply pressure is measured to be outside of predetermined limits.

Referring now to FIG. 20, there is shown the pump unit 1140 immediately prior to closing with cover 1103. In this embodiment, cover 1103 attaches to housing 1104 by a snap fitting arrangement including a pair of rigid lugs on one side (as best seen in FIG. 19) and a flexible lug 1162 on the opposed side of cover 1103 which snap fits into a complementary recess located on housing 1104. In order to remove cover 1103, a pin is inserted into aperture 1108 and operated to depress and release flexible lug 1162. This arrangement provides a substantially tamper proof housing preventing potential patient access to the pump mechanism and control electronics.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A garment for providing intravenous fluid delivery to a patient, the garment operable to be worn adjacent to the skin of the patient and including:
a pump unit support portion to support a portable infusion pump;
a fluid reservoir support portion to support a fluid reservoir carrying fluid for intravenous delivery to the patient via the infusion pump; and
at least one biological sensor carrying portion for carrying a biological sensor operative to measure a biological condition of the patient.

2. The garment of claim 1, wherein the garment is sized and shaped to be tight-fitting to be wearable under other garments

3. The garment of claim 2, wherein the garment is configured to worn on an upper body of the patient.

4. (canceled)

5. The garment of claim 3, wherein the pump unit support portion is located to be on a front of the upper body.

6. The garment of claim 3, wherein the fluid reservoir support portion is located on a front or back of the upper body.

7. The garment of claim 6, wherein if the fluid reservoir support portion is located on a back of the upper body, the fluid reservoir support portion is positioned to overlie one of the thoracic spine and the lumbar spine, and if the fluid reservoir support portion is located on a front of the upper body, the fluid reservoir support portion is positioned to overlie one or more of the abdomen and the thorax.

8. (canceled)

9. The garment of claim 1, wherein the at least one biological sensor carrying portion is for carrying a sensor to sense at least one of: temperature; heart rate; pulse; respiratory rate; electrocardiogram signals; respiratory noise; blood pressure and blood oxygen saturation.

10. The garment of claim 1, wherein the at least one biological sensor carrying portion includes a plurality of biological sensor carrying portions, the plurality of carrying portions arranged to carry respective temperatures sensors to in combination sense a core temperature of the patient.

11. The garment of claim 10, wherein the locations on the garment of the plurality of biological sensor carrying portions are selected from:
either side of the rib cage under the arm;
sternum; or
upper thoracic region.

12. The garment of claim 1, wherein the garment is at least partly stretchable and comprises moisture transmissive materials in at least some parts of the garment that are to overlie the skin.

13. A system for providing intravenous fluid delivery to a patient, comprising:
a garment worn by the patient adjacent to the skin, the garment including:
a pump unit support portion supporting a portable infusion pump;
a fluid reservoir support portion supporting a fluid reservoir, wherein the fluid reservoir is operably connected to the portable infusion pump by a fluid supply conduit to pump fluid from the fluid reservoir for intravenous delivery to a delivery site; and
at least one biological sensor for sensing a biological condition of the patient, the at least one biological sensor carried by a respective biological sensor carrying portion forming part of the garment.

14. The system of claim 13, wherein the garment is sized and shaped to be tight-fitting to be wearable under other garments.

15. The garment of claim 14, wherein the garment is configured to worn on an upper body of the patient.

16. (canceled)

17. The system of claim 13, wherein the fluid supply conduit is incorporated into the garment.

18. (canceled)

19. The system of claim 13, where the at least one sensor is for sensing at least one of: temperature; heart rate; pulse; respiratory rate; electrocardiogram signals; respiratory noise; blood pressure and blood oxygen saturation.

20. The system of claim 13, wherein the garment includes a plurality of biological sensor carrying portions, the plurality of biological sensor carrying portions arranged to carry respective temperatures sensors to in combination sense a core temperature of the patient.

21. The system of claim 20, wherein the locations of the plurality of biological sensor carrying portions are selected from:
either side of the rib cage under the arm;
sternum; or
upper thoracic region.

22. The system of claim 13, wherein sensing and status information from the at least one biological sensor is wirelessly or directly communicated to any one of:
a controller for monitoring and controlling the operation of the portable infusion pump;
a transceiver device; or
a handheld computing device.

23. The system of claim 22, wherein any one of the controller for the portable infusion pump, the transceiver device or the handheld computing device, having received the sensing and status information from the at least one biological sensor, is configured to then determine an alarm condition based on this sensing and status information.

24. The system of claim 23, wherein the alarm condition indicates any one of variation in body temperature, heart rate or respiratory noise level of the patient.

25. (canceled)

26. (canceled)

27. (canceled)

28. (canceled)

29. (canceled)
